(19) United States
(12) Patent Application Publication
(10) Pub. No.: US 2023/0041743 A1
(43) Pub. Date: Feb. 9, 2023

(54) 1,8-NAPHTHYRIDIN-2-ONE COMPOUNDS FOR THE TREATMENT OF AUTOIMMUNE DISEASE

(71) Applicant: Hoffmann-La Roche Inc., Little Falls, NJ (US)

(72) Inventors: Hong SHEN, Shanghai (CN); Xiaoqing WANG, Shanghai (CN); Hongtao XU, Shanghai (CN); Zhisen ZHANG, Shanghai (CN); Wei ZHU, Shanghai (CN); Ge ZOU, Shanghai (CN)

(73) Assignee: Hoffmann-La Roche Inc., Little Falls, NJ (US)

(21) Appl. No.: 17/780,123

(22) PCT Filed: Nov. 24, 2020
(86) PCT No.: PCT/EP2020/083113

§ 371 Date: May 26, 2022

(30) Foreign Application Priority Data
Nov. 26, 2019 (CN) PCT/CN2019/120996

(51) Int. Cl. C07D 519/00

(52) U.S. Cl. CPC C07D 519/00

(57) ABSTRACT

The present invention relates to compounds of formula (I), wherein R¹ to R³, m and n are as described herein, and their pharmaceutically acceptable salt, enantiomer or diastereomer thereof, and compositions including the compounds and methods of using the compounds.

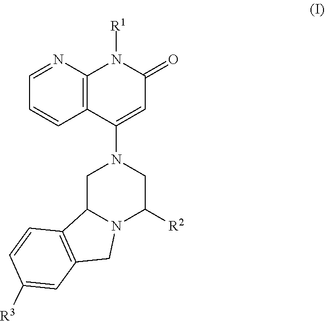

The present invention relates to organic compounds useful for therapy and/or prophylaxis in a mammal, and in particular to antagonist of TLR7 and/or TLR8 and/or TLR9 useful for treating systemic lupus erythematosus or lupus nephritis.

### FIELD OF THE INVENTION

Autoimmune connective tissue disease (CTD) include prototypical autoimmune syndromes such as Systemic Lupus Erythematosus (SLE), primary Sjögren's syndrome (pSjS), mixed connective tissue disease (MCTD), Dermatomyositis/Polymyositis (DM/PM), Rheumatoid Arthritis (RA), and systemic sclerosis (SSc). With the exception of RA, no really effective and safe therapies are available to patients. SLE represents the prototypical CTD with a prevalence of 20-150 per 100,000 and causes broad inflammation and tissue damage in distinct organs, from commonly observed symptoms in the skin and joints to renal, lung, or heart failure. Traditionally, SLE has been treated with nonspecific anti-inflammatory or immunosuppressive drugs. However, long term usage of immunosuppressive drug, e.g. corticosteroids is only partially effective, and is associated with undesirable toxicity and side effects. Belimumab is the only FDA-approved drug for lupus in the last 50 years, despite its modest and delayed efficacy in only a fraction of SLE patients (Navarra, S. V. et al Lancet 2011, 377, 721.). Other biologics, such as anti-CD20 mAbs, mAbs against or soluble receptors of specific cytokines, have failed in most clinical studies. Thus, novel therapies are required that provide sustained improvement in a greater proportion of patient groups and are safer for chronic use in many autoimmune as well as auto-inflammation diseases.

Toll Like Receptors (TLR) are an important family of pattern recognition receptors (PRR) which can initiate broad immune responses in a wide variety of immune cells. As natural host defense sensors, endosomal TLRs 7, 8 and 9 recognize nucleic acids derived from viruses, bacteria; specifically, TLR7/8 and TLR9 recognize single-stranded RNA (ssRNA) and single-stranded CpG-DNA, respectively. However, aberrant nucleic acid sensing of TRL7,8,9 is considered as a key node in a broad of autoimmune and auto-inflammatory diseases (Krieg, A. M. et al. Immunol. Rev. 2007, 220, 251. Jimenez-Dalmaroni, M. J. et al Autoimmun Rev. 2016, 15, 1. Chen, J. Q., et al. Clinical Reviews in Allergy & Immunology 2016, 50, 1.). Anti-RNA and anti-DNA antibodies are well established diagnostic markers of SLE, and these antibodies can deliver both self-RNA and self-DNA to endosomes. While self-RNA complexes can be recognized by TLR7 and TLR8, self-DNA complexes can trigger TLR9 activation. Indeed, defective clearance of self-RNA and self-DNA from blood and/or tissues is evident in SLE (Systemic Lupus Erythematosus) patients. TLR7 and TLR9 have been reported to be upregulated in SLE tissues, and correlate with chronicity and activity of lupus nephritis, respectively. In B cells of SLE patients, TLR7 expression correlates with anti-RNP antibody production, while TLR9 expression with IL-6 and anti-dsDNA antibody levels. Consistently, in lupus mouse models, TLR7 is required for anti-RNA antibodies, and TLR9 is required for anti-nucleosome antibody. On the other hand, overexpression of TLR7 or human TLR8 in mice promotes autoimmunity and autoinflammation. Moreover, activation of TLR8 specifically contributes to inflammatory cytokine secretion of mDC/macrophages, neutrophil NETosis, induction of Th17 cells, and suppression of Treg cells. In addition to the described role of TLR9 in promoting autoantibody production of B cells, activation of TLR9 by self-DNA in pDC also leads to induction of type I IFNs and other inflammatory cytokines. Given these roles of TLR9 in both pDC and B cells, both as key contributors to the pathogenesis of autoimmune diseases, and the extensive presence of self-DNA complexes that could readily activate TLR9 in many patients with autoimmune diseases, it may have extra benefit to further block self-DNA mediated TLR9 pathways on top of inhibition of TLR7 and TLR8 pathways. Taken together, TLR7, 8, and 9 pathways represent new therapeutic targets for the treatment of autoimmune and auto-inflammatory diseases, for which no effective steroid-free and non-cytotoxic oral drugs exist, and inhibition of all these pathways from the very upstream may deliver satisfying therapeutic effects. As such, we invented oral compounds that target and suppress TLR7, TLR8 and TLR9 for the treatment of autoimmune and auto-inflammatory diseases.

### SUMMARY OF THE INVENTION

The present invention relates to novel compounds of formula (I) and (Ia),

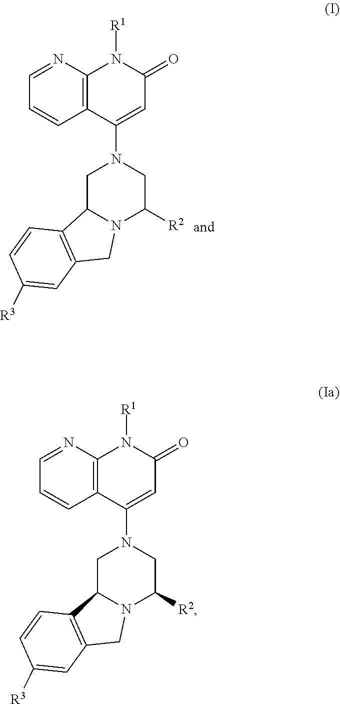

wherein
R¹ is C₁₋₆alkyl;
R² is C₁₋₆alkyl;
R³ is (C₁₋₆alkoxyC₁₋₆alkyl)piperazinyl; (C₁₋₆alkyl)₂aminoC₁₋₆alkoxy; 2,5-diazabicyclo[2.2.1]heptanyl; 3,4,4a,5,7,7a-hexahydro-2H-pyrrolo[3,4-b][1,4]oxazinyl; 3,8-diazabicyclo[3.2.1]octanyl; 3-oxa-9-azabicyclo[3.3.1]nonanyl; 5-oxa-2,8-diazaspiro[3.5]nonanyl; amino(C₁₋₆alkoxy)piperidinyl; amino(C₁₋₆alkoxy)pyrrolidinyl; amino(C₁₋₆alkyl)azetidinyl; amino(C₁₋₆alkyl)piperidinyl; amino(C₁₋₆alkyl)pyrrolidinyl; amino-1,4-oxazepanyl; aminohalopyrrolidinyl; aminopiperidinyl; C₁₋₆alkyl-2,6-diazaspiro[3.3]heptanyl; C₁₋₆alkylpiperazinyl; morpholinylC₃₋₇cycloalkyl; piperazinyl; piperidinyl or pyrrolidinyl;
   or a pharmaceutically acceptable salt thereof.

Another object of the present invention is related to novel compounds of formula (I) or (Ia), their manufacture, medicaments based on a compound in accordance with the invention and their production as well as the use of compounds of formula (I) or (Ia) as TLR7 and/or TLR8 and/or TLR9 antagonist, and for the treatment or prophylaxis of systemic lupus erythematosus or lupus nephritis. The compounds of formula (I) or (Ia) show superior TLR7 and/or TLR8 and/or TLR9 antagonism activity. In addition, the compounds of formula (I) or (Ia) also show good hPBMC, cytotoxicity, solubility, human microsome stability and SDPK profiles, as well as low CYP inhibition.

### DETAILED DESCRIPTION OF THE INVENTION

### Definitions

The term “C₁₋₆alkyl” denotes a saturated, linear or branched chain alkyl group containing 1 to 6, particularly 1 to 4 carbon atoms, for example methyl, ethyl, n-propyl, isopropyl, n-butyl, isobutyl, tert-butyl and the like. Particular “C₁₋₆alkyl” groups are methyl, ethyl and n-propyl.

The term “C₃₋₇cycloalkyl” denotes a saturated carbon ring containing from 3 to 7 carbon atoms, particularly from 3 to 6 carbon atoms, for example, cyclopropyl, cyclobutyl, cyclopentyl, cyclohexyl, cycloheptyl and the like. Particular “C₃₋₇cycloalkyl” groups are cyclopropyl, cyclopentyl and cyclohexyl.

The term “halogen” and “halo” are used interchangeably herein and denote fluoro, chloro, bromo, or iodo.

The term “C₁₋₆alkoxy” denotes C₁₋₆alkyl-O—.

The term “halopyrrolidinyl” denotes a pyrrolidinyl substituted once, twice or three times by halogen. Examples of halopyrrolidinyl include, but not limited to, difluoropyrrolidinyl and fluoropyrrolidinyl.

The term “pharmaceutically acceptable salts” denotes salts which are not biologically or otherwise undesirable. Pharmaceutically acceptable salts include both acid and base addition salts.

The term “pharmaceutically acceptable acid addition salt” denotes those pharmaceutically acceptable salts formed with inorganic acids such as hydrochloric acid, hydrobromic acid, sulfuric acid, nitric acid, carbonic acid, phosphoric acid, and organic acids selected from aliphatic, cycloaliphatic, aromatic, araliphatic, heterocyclic, carboxylic, and sulfonic classes of organic acids such as formic acid, acetic acid, propionic acid, glycolic acid, gluconic acid, lactic acid, pyruvic acid, oxalic acid, malic acid, maleic acid, maloneic acid, succinic acid, fumaric acid, tartaric acid, citric acid, aspartic acid, ascorbic acid, glutamic acid, anthranilic acid, benzoic acid, cinnamic acid, mandelic acid, embonic acid, phenylacetic acid, methanesulfonic acid, ethanesulfonic acid, p-toluenesulfonic acid, and salicyclic acid.

The term “pharmaceutically acceptable base addition salt” denotes those pharmaceutically acceptable salts formed with an organic or inorganic base. Examples of acceptable inorganic bases include sodium, potassium, ammonium, calcium, magnesium, iron, zinc, copper, manganese, and aluminum salts. Salts derived from pharmaceutically acceptable organic nontoxic bases includes salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, trimethamine, dicyclohexylamine, lysine, arginine, histidine, caffeine, procaine, hydrabamine, choline, betaine, ethylenediamine, glucosamine, methylglucamine, theobromine, purines, piperizine, piperidine, N-ethylpiperidine, and polyamine resins.

The term “A pharmaceutically active metabolite” denotes a pharmacologically active product produced through metabolism in the body of a specified compound or salt thereof. After entry into the body, most drugs are substrates for chemical reactions that may change their physical properties and biologic effects. These metabolic conversions, which usually affect the polarity of the compounds of the invention, alter the way in which drugs are distributed in and excreted from the body. However, in some cases, metabolism of a drug is required for therapeutic effect.

The term “therapeutically effective amount” denotes an amount of a compound or molecule of the present invention that, when administered to a subject, (i) treats or prevents the particular disease, condition or disorder, (ii) attenuates, ameliorates or eliminates one or more symptoms of the particular disease, condition, or disorder, or (iii) prevents or delays the onset of one or more symptoms of the particular disease, condition or disorder described herein. The therapeutically effective amount will vary depending on the compound, the disease state being treated, the severity of the disease treated, the age and relative health of the subject, the route and form of administration, the judgement of the attending medical or veterinary practitioner, and other factors.

The term “pharmaceutical composition” denotes a mixture or solution comprising a therapeutically effective amount of an active pharmaceutical ingredient together with pharmaceutically acceptable excipients to be administered to a mammal, e.g., a human in need thereof.

Antagonist OF TLR7 and/or TLR8 and/or TLR9

The present invention relates to (i) a compound of formula (I),

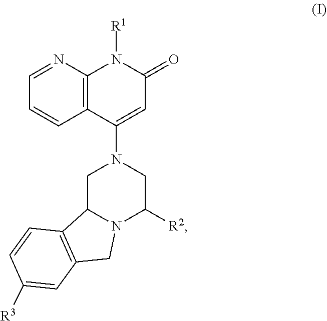

wherein
R¹ is C₁₋₆alkyl;
R² is C₁₋₆alkyl;
R³ is (C₁₋₆alkoxyC₁₋₆alkyl)piperazinyl; (C₁₋₆alkyl)₂aminoC₁₋₆alkoxy; 2,5-diazabicyclo[2.2.1]heptanyl; 3,4,4a,5,7,7a-hexahydro-2H-pyrrolo[3,4-b][1,4]oxazinyl; 3,8-diazabicyclo[3.2.1]octanyl; 3-oxa-9-azabicyclo[3.3.1]nonanyl; 5-oxa-2,8-diazaspiro[3.5]nonanyl; amino(C₁₋₆alkoxy)piperidinyl; amino(C₁₋₆alkoxy)pyrrolidinyl; amino(C₁₋₆alkyl)azetidinyl; amino(C₁₋₆alkyl)piperidinyl; amino(C₁₋₆alkyl)pyrrolidinyl; amino-1,4-oxazepanyl; aminohalopyrrolidinyl; aminopiperidinyl; C₁₋₆alkyl-2,6-diazaspiro[3.3]heptanyl; C₁₋₆alkylpiperazinyl; morpholinylC₃₋₇cycloalkyl; piperazinyl; piperidinyl or pyrrolidinyl;
   or a pharmaceutically acceptable salt thereof.

Another embodiment of present invention is (ii) a compound of formula (Ia),

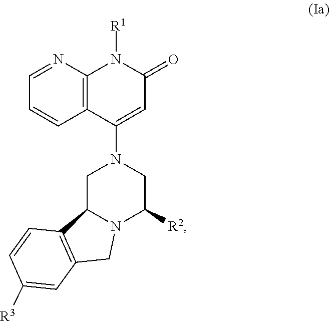

wherein
R¹ is C₁₋₆alkyl;
R² is C₁₋₆alkyl;
R³ is (C₁₋₆alkoxyC₁₋₆alkyl)piperazinyl; (C₁₋₆alkyl)₂aminoC₁₋₆alkoxy; 2,5-diazabicyclo[2.2.1]heptanyl; 3,4,4a,5,7,7a-hexahydro-2H-pyrrolo[3,4-b][1,4]oxazinyl; 3,8-diazabicyclo[3.2.1]octanyl; 3-oxa-9-azabicyclo[3.3.1]nonanyl; 5-oxa-2,8-diazaspiro[3.5]nonanyl; amino(C₁₋₆alkoxy)piperidinyl; amino(C₁₋₆alkoxy)pyrrolidinyl; amino(C₁₋₆alkyl)azetidinyl; amino(C₁₋₆alkyl)piperidinyl; amino(C₁₋₆alkyl)pyrrolidinyl; amino-1,4-oxazepanyl; aminohalopyrrolidinyl; aminopiperidinyl; C₁₋₆alkyl-2,6-diazaspiro[3.3]heptanyl; C₁₋₆alkylpiperazinyl; morpholinylC₃₋₇cycloalkyl; piperazinyl; piperidinyl or pyrrolidinyl;
   or a pharmaceutically acceptable salt thereof.

A further embodiment of present invention is (iii) a compound of formula (I) or (Ia) according to (i) or (ii), or a pharmaceutically acceptable salt thereof, wherein
R¹ is methyl or ethyl;
R² is methyl;
R³ is 2-(dimethylamino)ethoxy; 2-(methoxymethyl)piperazin-1-yl; 2,5-diazabicyclo[2.2.1]heptan-2-yl; 2-methylpiperazin-1-yl; 3,4,4a,5,7,7a-hexahydro-2H-pyrrolo[3,4-b][1,4]oxazin-6-yl; 3,8-diazabicyclo[3.2.1]octan-3-yl; 3-amino-1-piperidinyl; 3-amino-3-methyl-1-piperidinyl; 3-amino-3-methyl-azetidin-1-yl; 3-amino-3-methyl-pyrrolidin-1-yl; 3-amino-4-fluoro-pyrrolidin-1-yl; 3-amino-4-methoxy-1-piperidinyl; 3-amino-4-methoxy-pyrrolidin-1-yl; 3-methylpiperazin-1-yl; 3-oxa-9-azabicyclo[3.3.1]nonan-7-yl; 3-piperidinyl; 4-amino-3-methoxy-1-piperidinyl; 4-amino-4-methyl-1-piperidinyl; 4-morpholinocyclohexyl; 4-piperidinyl; 5-oxa-2,8-diazaspiro[3.5]nonan-2-yl; 5-oxa-2,8-diazaspiro[3.5]nonan-8-yl; 6-amino-1,4-oxazepan-4-yl; 6-methyl-2,6-diazaspiro[3.3]heptan-2-yl; piperazin-1-yl or pyrrolidin-3-yl;
   or a pharmaceutically acceptable salt thereof.

A further embodiment of present invention is (iv) a compound of formula (I) or (Ia) according to any one of (i) to (iii), or a pharmaceutically acceptable salt thereof, wherein R³ is amino(C₁₋₆alkoxy)pyrrolidinyl; amino(C₁₋₆alkyl)piperidinyl; amino-1,4-oxazepanyl; aminopiperidinyl; C₁₋₆alkyl-2,6-diazaspiro[3.3]heptanyl; C₁₋₆alkylpiperazinyl; C₁₋₆alkylpiperazinyl; morpholinylC₃₋₇cycloalkyl; piperazinyl; piperidinyl or 3-oxa-9-azabicyclo[3.3.1]nonanyl.

A further embodiment of present invention is (v) a compound of formula (I) or (Ia) according to any one of (i) to (iv), or a pharmaceutically acceptable salt thereof, wherein R³ is 3-amino-1-piperidinyl; 3-amino-3-methyl-1-piperidinyl; 3-amino-4-methoxy-pyrrolidin-1-yl; 3-methylpiperazin-1-yl; 4-amino-4-methyl-1-piperidinyl; 4-morpholinocyclohexyl; 4-piperidinyl; 6-amino-1,4-oxazepan-4-yl; 6-methyl-2,6-diazaspiro[3.3]heptan-2-yl; piperazin-1-yl or 3-oxa-9-azabicyclo[3.3.1]nonan-7-yl.

A further embodiment of present invention is (vi) a compound of formula (I) or (Ia) according to any one of (i) to (v), or a pharmaceutically acceptable salt thereof, wherein
R¹ is C₁₋₆alkyl;
R² is C₁₋₆alkyl;
R³ is amino(C₁₋₆alkoxy)pyrrolidinyl; amino(C₁₋₆alkyl)piperidinyl; amino-1,4-oxazepanyl; aminopiperidinyl; C₁₋₆alkyl-2,6-diazaspiro[3.3]heptanyl; C₁₋₆alkylpiperazinyl; C₁₋₆alkylpiperazinyl; morpholinylC₃₋₇cycloalkyl; piperazinyl; piperidinyl or 3-oxa-9-azabicyclo[3.3.1]nonanyl;
   or a pharmaceutically acceptable salt thereof.

A further embodiment of present invention is (vii) a compound of formula (I) or (Ia) according to any one of (i) to (vi), or a pharmaceutically acceptable salt thereof, wherein
R¹ is methyl;
R² is methyl;
R³ is 3-amino-1-piperidinyl; 3-amino-3-methyl-1-piperidinyl 3-amino-4-methoxy-pyrrolidin-1-yl; 3-methylpiperazin-1-yl; 4-amino-4-methyl-1-piperidinyl; 4-morpholinocyclohexyl; 4-piperidinyl; 6-amino-1,4-oxazepan-4-yl; 6-methyl-2,6-diazaspiro[3.3]heptan-2-yl; piperazin-1-yl or 3-oxa-9-azabicyclo[3.3.1]nonan-7-yl;
   or a pharmaceutically acceptable salt thereof.

Another embodiment of present invention is that (viii) compounds of formula (I) or (Ia) are selected from the following:
4-[(4R,10bS)-8-(3-amino-3-methyl-azetidin-1-yl)-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(6-methyl-2,6-diazaspiro[3.3]heptan-2-yl)-3,4,6,10b-tetrahydro-1H-pyrazino[2,1-a]isoindol-2-yl]-1-methyl-1,8-naphthyridin-2-one;
4-[(4R,10bS)-4-methyl-8-(5-oxa-2,8-diazaspiro[3.5]nonan-2-yl)-3,4,6,10b-tetrahydro-1H-pyrazino[2,1-a]isoindol-2-yl]-1-methyl-1,8-naphthyridin-2-one;
4-[(4R,10bS)-8-[(3R)-3-amino-3-methyl-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(4aR,7aR)-3,4,4a,5,7,7a-hexahydro-2H-pyrrolo[3,4-b][1,4]oxazin-6-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R,4S)-3-amino-4-fluoro-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3S,4S)-4-amino-3-methoxy-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3S,4S)-3-amino-4-methoxy-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(2S)-2-(methoxymethyl)piperazin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(5-oxa-2,8-diazaspiro[3.5]nonan-8-yl)-3,4,6,10b-tetrahydro-1H-pyrazino[2,1-a]isoindol-2-yl]-1-methyl-1,8-naphthyridin-2-one;
4-[(4R,10bS)-4-methyl-8-piperazin-1-yl-3,4,6,10b-tetrahydro-1H-pyrazino[2,1-a]isoindol-2-yl]-1-methyl-1,8-naphthyridin-2-one;
4-[(4R,10bS)-4-methyl-8-[(3S)-3-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-4-methyl-8-[(3R)-3-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-8-[(3S)-3-amino-3-methyl-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R)-3-amino-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-[(2R)-2-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-8-(4-amino-4-methyl-1-piperidyl)-4-methyl-3,4,6,10b-tetrahydro-1H-pyrazino[2,1-a]isoindol-2-yl]-1-methyl-1,8-naphthyridin-2-one;
4-[(4R,10bS)-8-[(1S,4S)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(1R,4R)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one;
4-[(4R,10bS)-8-(3,8-diazabicyclo[3.2.1]octan-3-yl)-4-methyl-3,4,6,10b-tetrahydro-1H-pyrazino[2,1-a]isoindol-2-yl]-1-methyl-1,8-naphthyridin-2-one;
4-[(4R,10bS)-8-[(6R)-6-amino-1,4-oxazepan-4-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[2-(dimethylamino)ethoxy]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(4-piperidyl)-3,4,6,10b-tetrahydro-1H-pyrazino[2,1-a]isoindol-2-yl]-1-methyl-1,8-naphthyridin-2-one;
4-[(4R,10bS)-4-methyl-8-(3-piperidyl)-3,4,6,10b-tetrahydro-1H-pyrazino[2,1-a]isoindol-2-yl]-1-methyl-1,8-naphthyridin-2-one;
4-[(4R,10bS)-4-methyl-8-pyrrolidin-3-yl-3,4,6,10b-tetrahydro-1H-pyrazino[2,1-a]isoindol-2-yl]-1-methyl-1,8-naphthyridin-2-one;
4-[(4R,10bS)-4-methyl-8-(trans-4-morpholinocyclohexyl)-3,4,6,10b-tetrahydro-1H-pyrazino[2,1-a]isoindol-2-yl]-1-methyl-1,8-naphthyridin-2-one;
4-[(4R,10bS)-4-methyl-8-(cis-4-morpholinocyclohexyl)-3,4,6,10b-tetrahydro-1H-pyrazino[2,1-a]isoindol-2-yl]-1-methyl-1,8-naphthyridin-2-one;
4-[(4R,10bS)-4-methyl-8-(endo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one;
4-[(4R,10bS)-4-methyl-8-(exo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one; and
4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-ethyl-1,8-naphthyridin-2-one;
   or a pharmaceutically acceptable salt, enantiomer or diastereomer thereof.

### Synthesis

The compounds of the present invention can be prepared by any conventional means. Suitable processes for synthesizing these compounds as well as their starting materials are provided in the schemes below and in the examples. All substituents, in particular, R¹ to R⁶ are as defined above unless otherwise indicated. Furthermore, and unless explicitly otherwise stated, all reactions, reaction conditions, abbreviations and symbols have the meanings well known to a person of ordinary skill in organic chemistry.

A general synthetic route for preparing the compound of formula (I) is shown in Scheme 1 below.

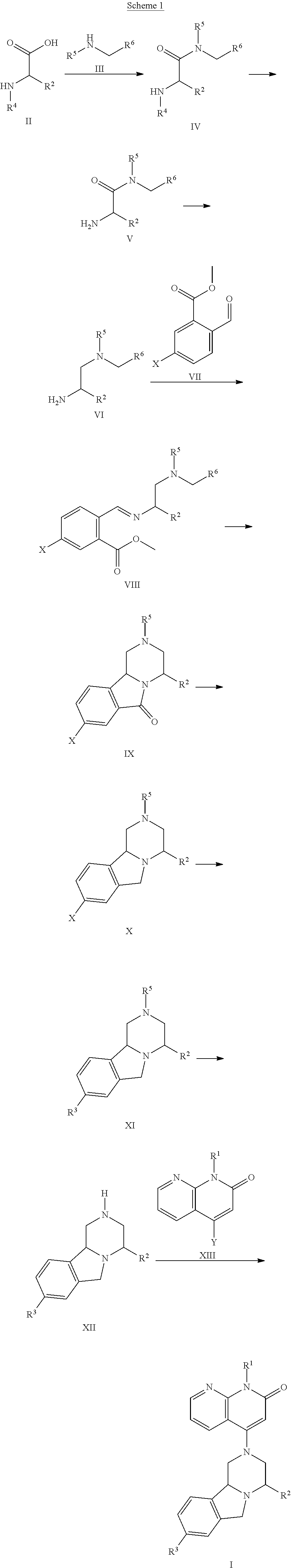

wherein X and Y are halogen or leaving group, for example, OTf or OMs; R⁴ and R⁵ are protecting groups, for example, R⁴ is Boc and R⁵ is benzyl; R⁶ is alkylsilyl, for example, trimethylsilyl.

The amide coupling of protected amino acid (II) and silylamine (III) can be achieved using coupling reagents, such as HATU and DIPEA, to afford intermediate (IV). After R⁴ is removed by selective deprotection, the amide bond in the resulting intermediate (V) can be reduced under reductive conditions, such as treatment of LAH, to afford diamine (VI). Imine (VIII), which can be formed by condensation of aldehyde (VII) and diamine (VI) under typical dehydration conditions, is cyclized under photo-redox conditions, which is catalyzed by blue light and Ir-based catalyst, such as [Ir(dtbbpy)(ppy)₂][PF6], to afford tricyclic lactam (IX). When treated with reducing reagent, such as LAH, lactam (IX) can be reduced to compound of formula (X). Compound of formula (X) can be used as a common intermediate for further functionalization under metal catalyzed coupling conditions, such as Buchwald-Hartwig amination, Suzuki coupling, Negishi coupling, Stille coupling, or Pd-catalyzed C═O insertion. For example, under Buchwald-Hartwig amination conditions (ref: Acc. Chem. Res. 1998, 31, 805-818; Chem. Rev. 2016, 116, 12564-12649; Topics in Current Chemistry, 2002, 219, 131-209; and references cited therein) with a catalyst, such as Ruphos Pd-G2, and a base, such as Cs₂CO₃, compound of formula (XI) can be generated from compound of formula (X). After selective deprotection of R⁵ group under typical conditions (e.g. removal of benzyl protecting group by hydrogenation over catalytic amount of Palladium on carbon), the resulting compound of formula (XII) can be submitted to nucleophilic aromatic substitution conditions (e.g. heating with halide (XIII) in the presence of DIEPA in DMSO), or Buchwald-Hartwig amination conditions (e.g. heating with halide (XIII) in the presence of a catalyst, such as Ruphos Pd-G2, and a base, such as Cs₂CO₃, to afford compound of formula (I) or (Ia). In some embodiment, the compound of formula (XII) may contain a protecting group, e.g. Boc, which will be removed before affording the final compound of formula (I) or (Ia).

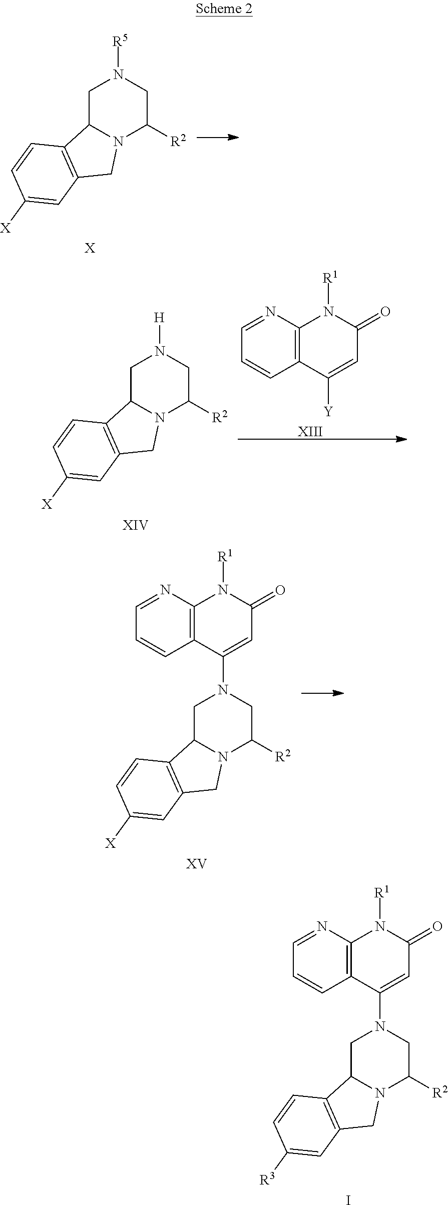

Alternatively, as shown in Scheme 2, after R⁵ is removed from formula (X) by selective deprotection, the resulting compound of formula (XIV) can react with halide (XIII) to afford compound of formula (XV) by nucleophilic aromatic substitution in the presence of a base, such as DIEPA. Compound of formula (I) or (Ia) can be obtained from compound of formula (XV) via metal catalyzed coupling conditions: Buchwald-Hartwig amination in the presence of a catalyst, such as Ruphos Pd-G2, and a base, such as Cs₂CO₃; Suzuki coupling with R³-boronic acid or R³-boronic ester, in the presence of a palladium catalyst, such as tetrakis(triphenylphosphine)palladium(0) or [1,1′-bis(diphenylphosphino)ferrocene]dichloropalladium (II), complex with dichloromethane and a base, such as potassium carbonate in solvent; Stille coupling with organotin reagent, in the presence of a palladium(0) catalyst, such as tetrakis(triphenylphosphine)palladium(0); or Negishi coupling with organozinc reagent in the presence of a palladium(0) catalyst, such as tetrakis(triphenylphosphine)palladium(0) or [1,1′-bis(diphenylphosphino)ferrocene]dichloropalladium (II). In some embodiment, the compound of formula (XII) may contain a protecting group, e.g. Boc, which will be removed before affording the final compound of formula (I) or (Ia).

Compounds of this invention can be obtained as mixtures of diastereomers or enantiomers, which can be separated by methods well known in the art, e.g. (chiral) HPLC or SFC.

This invention also relates to a process for the preparation of a compound of formula (I) or (Ia) comprising any of the following steps:
a) the substitution reaction or Buchwald-Hartwig amination of compound of formula (XII),

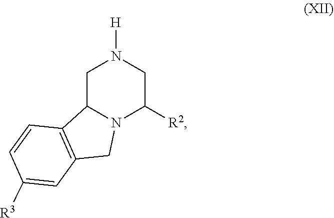

and compound of formula (XIII),

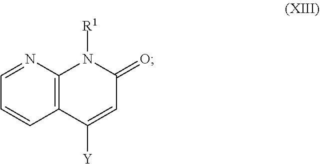

b) the Buchwald-Hartwig amination reaction of compound of formula (XV),

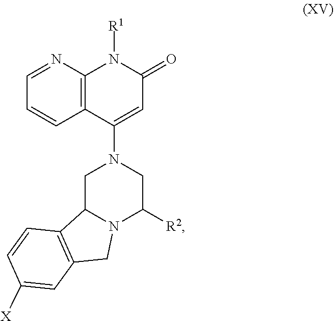

and amine HR³; or Suzuki coupling reaction between compound of formula (IX) and R³-boronic acid or R³-boronic ester;

A compound of formula (I) or (Ia) when manufactured according to the above process with achiral or chiral starting materials is also an object of the invention.

### Indications and Methods of Treatment

The present invention provides compounds that can be used as TLR7 and/or TLR8 and/or TLR9 antagonist, which inhibits pathway activation through TLR7 and/or TLR8 and/or TLR9 as well as respective downstream biological events including, but not limited to, innate and adaptive immune responses mediated through the production of all types of cytokines and all forms of auto-antibodies. Accordingly, the compounds of the invention are useful for blocking TLR7 and/or TLR8 and/or TLR9 in all types of cells that express such receptor(s) including, but not limited to, plasmacytoid dendritic cell, B cell, T cell, macrophage, monocyte, neutrophil, keratinocyte, epithelial cell. As such, the compounds can be used as a therapeutic or prophylactic agent for systemic lupus erythematosus and lupus nephritis.

The present invention provides methods for treatment or prophylaxis of systemic lupus erythematosus and lupus nephritis in a patient in need thereof.

Another embodiment includes a method of treating or preventing systemic lupus erythematosus and lupus nephritis in a mammal in need of such treatment, wherein the method comprises administering to said mammal a therapeutically effective amount of a compound of formula (I), a stereoisomer, tautomer, prodrug or pharmaceutically acceptable salt thereof.

### EXAMPLES

The invention will be more fully understood by reference to the following examples. They should not, however, be construed as limiting the scope of the invention.

### ABBREVIATIONS

The invention will be more fully understood by reference to the following examples. They should not, however, be construed as limiting the scope of the invention.

Abbreviations used herein are as follows:
ACN: acetonitrile
DCM: dichloromethane
DIPEA diethylisopropylamine
EA or EtOAc: ethyl acetate
FA: formic acid
HATU 1-[Bis(dimethylamino)methylene]-1H-1,2,3-triazolo[4,5-b]pyridinium 3-oxid hexafluorophosphate
hr hour
hrs hours
IC₅₀: half inhibition concentration
MS: mass spectrometry
prep-HPLC: preparative high performance liquid chromatography
RT: retention time
RuPhos Pd G2: chloro(2-dicyclohexylphosphino-2′,6′-diisopropoxy-1,1′-biphenyl)[2-(2′-amino-1,1′-biphenyl)]palladium(II) 2nd generation
SFC: supercritical fluid chromatography
TFA: trifluoroacetic acid
v/v volume ratio

General Experimental Conditions

Intermediates and final compounds were purified by flash chromatography using one of the following instruments: i) Biotage SP1 system and the Quad 12/25 Cartridge module. ii) ISCO combi-flash chromatography instrument. Silica gel brand and pore size: i) KP-SIL 60 A, particle size: 40-60 μm; ii) CAS registry NO: Silica Gel: 63231-67-4, particle size: 47-60 micron silica gel; iii) ZCX from Qingdao Haiyang Chemical Co., Ltd, pore: 200-300 or 300-400.

Intermediates and final compounds were purified by preparative HPLC on reversed phase column using XBridge™ Prep-C18 (5 μm, OBD™ 30×100 mm) column, SunFire™ Prep-C18 (5 μm, OBD™ 30×100 mm) column, Phenomenex Synergi-C18 (10 μm, 25×150 mm) or Phenomenex Gemini-C18 (10 μm, 25×150 mm). Waters AutoP purification System (Sample Manager 2767, Pump 2525, Detector: Micromass ZQ and UV 2487, solvent system: ACN and 0.1% ammonium hydroxide in water; ACN and 0.1% FA in water or ACN and 0.1% TFA in water). Or Gilson-281 purification System (Pump 322, Detector: UV 156, solvent system: ACN and 0.05% ammonium hydroxide in water; ACN and 0.225% FA in water; ACN and 0.05% HCl in water; ACN and 0.075% TFA in water; or ACN and water).

For SFC chiral separation, intermediates were separated by chiral column (Daicel chiralpak IC, 5 μm, 30×250 mm), AS (10 μm, 30×250 mm) or AD (10 μm, 30×250 mm) using Mettler Toledo Multigram III system SFC, Waters 80Q preparative SFC or Thar 80 preparative SFC, solvent system: CO₂ and IPA (0.5% TEA in IPA) or CO₂ and MeOH (0.1% NH₃—H₂O in MeOH), back pressure 100 bar, detection UV@ 254 or 220 nm.

LC/MS spectra of compounds were obtained using a LC/MS (Waters™ Alliance 2795-Micromass ZQ, Shimadzu Alliance 2020-Micromass ZQ or Agilent Alliance 6110-Micromass ZQ), LC/MS conditions were as follows (running time 3 or 1.5 mins):

Acidic condition I: A: 0.1% TFA in H₂O; B: 0.1% TFA in ACN;

Acidic condition II: A: 0.0375% TFA in H₂O; B: 0.01875% TFA in ACN;

Basic condition I: A: 0.1% NH₃—H₂O in H₂O; B: ACN;

Basic condition II: A: 0.025% NH₃—H₂O in H₂O; B: ACN;

Neutral condition: A: H₂O; B: ACN.

Mass spectra (MS): generally only ions which indicate the parent mass are reported, and unless otherwise stated the mass ion quoted is the positive mass ion (MH)⁺.

NMR Spectra were obtained using Bruker Avance 400 MHz.

The microwave assisted reactions were carried out in a Biotage Initiator Sixty microwave synthesizer. All reactions involving air-sensitive reagents were performed under an argon or nitrogen atmosphere. Reagents were used as received from commercial suppliers without further purification unless otherwise noted.

### PREPARATIVE EXAMPLES

The following examples are intended to illustrate the meaning of the present invention but should by no means represent a limitation within the meaning of the present invention:

### Intermediate A

### (4R,10bS)-2-benzyl-8-bromo-4-methyl-3,4,6,10b-tetrahydro-1H-pyrazino[2,1-a]isoindole

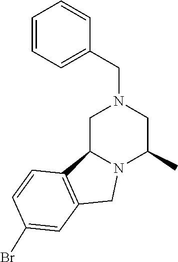 [SMILES: C[C@@H]1CN(Cc2ccccc2)C[C@@H]2c3ccc(Br)cc3CN12]

The titled compound was synthesized according to the following scheme:

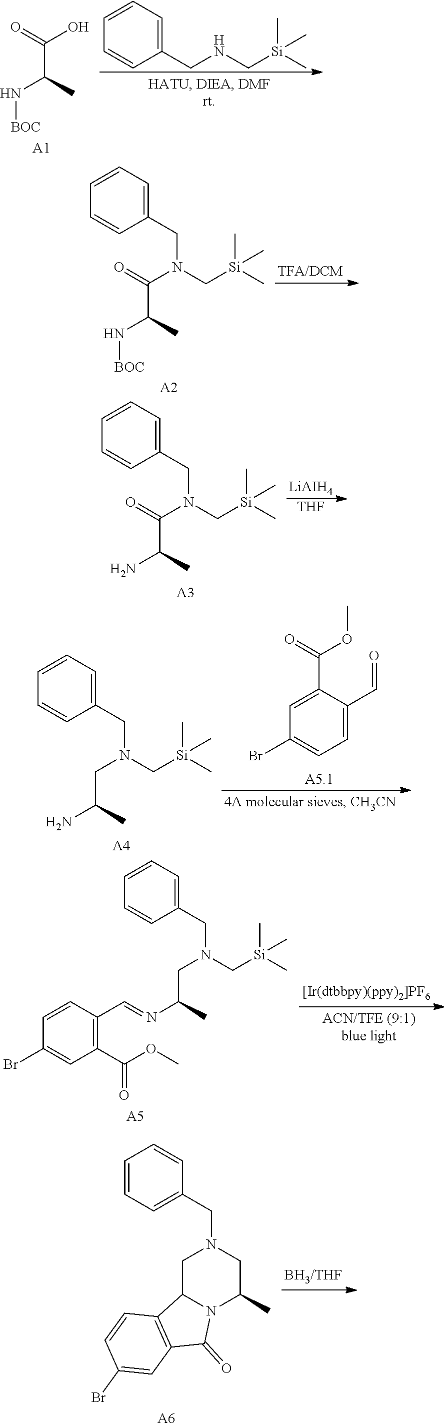

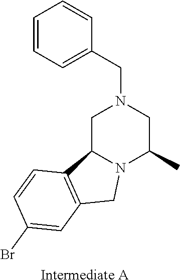

### Step 1: Preparation of tert-butyl N-[(1R)-2-[benzyl(trimethylsilylmethyl)amino]-1-methyl-2-oxo-ethyl]carbamate (compound A2)

To a solution of (2R)-2-(tert-butoxycarbonylamino)propanoic acid (compound A1, 10 g, 52.9 mmol) in DMF (40 mL) was added N-benzyl-1-(trimethylsilyl)methanamine (10.2 g, 52.9 mmol), HATU (20.1 g, 52.9 mmol) and DIEA (6.8 g, 9.2 mL, 52.9 mmol). The reaction mixture was stirred at room temperature overnight, then quenched with water (150 mL), and extracted with DCM (100 mL) for three times. The combined organic layer was washed with brine, dried over Na₂SO₄, filtered and concentrated in vacuo. The residue was purified by flash chromatography (silica gel, 40 g, 0% to 30% EtOAc in PE) to afford compound A2 (13.1 g, 68% yield). MS: calc'd 365 [(M+H)⁺], measured 365 [(M+H)⁺].

### Step 2: Preparation of (2R)-2-amino-N-benzyl-N-(trimethylsilylmethyl)propanamide (compound A3)

TFA (10 mL) was added to the solution of tert-butyl N-[(1R)-2-[benzyl(trimethylsilylmethyl)amino]-1-methyl-2-oxo-ethyl]carbamate (compound A2, 13.0 g, 35.7 mmol) in DCM (60 mL), and the mixture was stirred at room temperature for 4 hrs. The reaction was concentrated in vacuo, and the residue was partitioned with sat. NaHCO₃(aq) and EA. The organic layer was separated and the basic aqueous layer was extracted with DCM (80 mL) twice. The combined organic layer was dried over Na₂SO₄, filtered and concentrated in vacuo to afford compound A3 (9.1 g, 96% yield) without further purification. MS: calc'd 265 [(M+H)⁺], measured 265 [(M+H)⁺].

### Step 3: Preparation of (2R)—N1-benzyl-N1-(trimethylsilylmethyl)propane-1,2-diamine (compound A4)

To an ice-cooled solution of (2R)-2-amino-N-benzyl-N-(trimethylsilylmethyl)-propanamide (compound A3, 9.0 g, 34 mmol) in anhydrous THF (100 mL), LiAlH₄ (3.9 g, 102 mmol) was added slowly. After the addition was completed, the mixture was heated under reflux overnight. The reaction was cooled to room temperature, quenched with 20% NaOH(aq) before filtered and washed with EtOAc. The combined filtrate was concentrated in vacuo to afford compound A4 (5.7 g, 67% yield) without further purification. MS: calc'd 251 [(M+H)⁺], measured 251 [(M+H)⁺].

### Step 4: Preparation of (4R,10bS)-2-benzyl-8-bromo-4-methyl-1,3,4,10b-tetrahydro pyrazino[1,2-b]isoindol-6-one (compound A6)

A mixture of the (2R)—N1-benzyl-N1-(trimethylsilylmethyl)propane-1,2-diamine (compound A4, 3 g, 12 mmol), methyl 5-bromo-2-formylbenzoate (2.9 g, 12 mmol), and 4 A MS (5.0 g) in MeCN (80 mL) under N2 was stirred overnight at room temperature. The reaction was filtered through Celite and washed with DCM. The filtrate was concentrated in vacuo to afford the intermediate compound A5, the residue was re-dissolved in MeCN/TFE (45 mL/5 mL), followed by the addition of [Ir(dtbbpy)(ppy)₂][PF₆] (CAS: 676525-77-2, TCI, Catalog: D4887, 42.9 mg, 46.9 μmol). The resultant mixture was stirred at room temperature under the exposure of blue LEDs (synLED-16 A Discover, 12 W, wavelength 465-470 nm, purchased from SYNLED corp.) for 2 days. After the solvents were removed in vacuo, the residue was purified by flash chromatography (silica gel, 80 g, 20% to 70% EA in PE) to afford compound A6 (1.85 g, 42% yield). The stereochemistry was confirmed by NOESY. MS: calc'd 371, 373 [(M+H)⁺], measured 371, 373 [(M+H)⁺]. ¹H NMR (400 MHz, METHANOL-d₄) δ ppm 7.75 (d, J=1.71 Hz, 1H) 7.63 (dd, J=8.01, 1.77 Hz, 1H) 7.19-7.35 (m, 6H) 4.43 (dd, J=10.88, 3.67 Hz, 1H) 3.67-3.86 (m, 1H) 3.56 (s, 2H) 3.41-3.45 (m, 1H) 2.79-2.87 (m, 1H) 1.86 (t, J=11.07 Hz, 1H) 1.67 (d, J=6.97 Hz, 3H) 1.64 (t, J=11.07 Hz, 1H).

### Step 5: Preparation of (4R,10bS)-2-benzyl-8-bromo-4-methyl-3,4,6,10b-tetrahydro-1H-pyrazino[2,1-a]isoindole (Intermediate A)

A mixture of (4R,10bS)-2-benzyl-8-bromo-4-methyl-1,3,4,10b-tetrahydropyrazino[1,2-b]isoindol-6-one (compound A6, 1.9 g, 5.0 mmol) and BH3 solution (1M in THF, 40 mL, 40 mmol) was heated at 80° C. with stirring on for 5 hrs. HCl solution (6 N, 10 mL) was added slowly to the reaction mixture at 0° C. The resultant mixture was stirred at room temperature overnight, then the mixture was basified with a NaOH solution (2 N) to pH 10. The mixture was extracted with EtOAc twice. The combined organic layer was dried over MgSO₄, filtered and concentrated in vacuo. The residue was purified by flash chromatography (silica gel, 40 g, 30% to 100% EtOAc in PE) to afford Intermediate A (1.5 g, 85% yield). The stereochemistry was confirmed by NOESY. MS: calc'd 357 and 359 [(M+H)⁺], measured 357 and 359 [(M+H)⁺]. ¹H NMR (400 MHz, METHANOL-d₄) δ ppm 7.49 (s, 1H) 7.32-7.43 (m, 5H) 7.26-7.32 (m, 1H) 7.05 (d, J=7.95 Hz, 1H) 4.18 (d, J=12.59 Hz, 1H) 3.71 (br d, J=10.51 Hz, 1H) 3.55 (dd, J=12.47, 2.32 Hz, 1H) 3.36-3.31 (m, 1H) 2.97-2.89 (m, 1H) 2.77-2.87 (m, 1H) 2.12 (t, J=10.64 Hz, 1H) 2.00 (t, J=10.64 Hz, 1H) 1.14 (d, J=6.48 Hz, 3H).

### Intermediate B

### 4-Chloro-1-methyl-1,8-naphthyridin-2-one

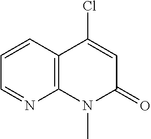

The titled compound was synthesized according to the following scheme:

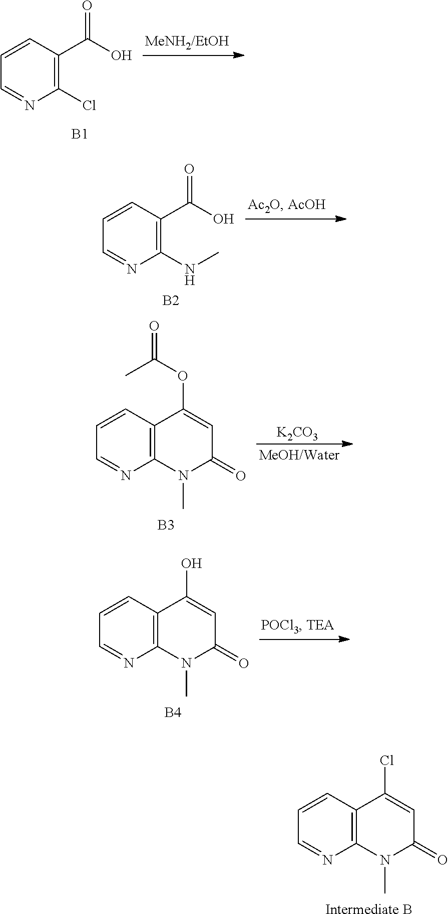

### Step (a): Preparation of 2-(methylamino)pyridine-3-carboxylic acid (compound B2)

2-chloronicotinic acid (compound B1, 1.0 kg, 6.3 mol) was dissolved in 33% monomethylamine (386.3 mol) solution in ethanol. The reaction mixture was stirred in the autoclave at 80° C. for 80 hrs, then concentrated in vacuo to afford compound A2.2 (1.4 kg, crude). MS: calc'd 153 [(M+H)⁺], measured 153 [(M+H)⁺].

### Step (b): Preparation of (1-methyl-2-oxo-1,8-naphthyridin-4-yl) acetate (compound B3)

A solution of 2-(methylamino)pyridine-3-carboxylic acid (compound B2, 1.4 kg, crude) in acetic anhydride (10.0 L, 105789 mmol) and Acetic acid (5.0 L) was heated to reflux for 2 hrs. The reaction mixture was concentrated in vacuo to afford compound B3 (1.8 kg, crude). MS: calc'd 219 [(M+H)⁺], measured 219 [(M+H)⁺].

### Step (c): Preparation of 4-hydroxy-1-methyl-1,8-naphthyridin-2-one (compound B4)

To a solution of (1-methyl-2-oxo-1,8-naphthyridin-4-yl) acetate (compound B3, 1.8 kg, crude) in methanol (12.0 L) was added a solution of potassium carbonate (1.9 kg, 13.7 mol) in water (3.6 L). The mixture was stirred at 25° C. for 2 hrs. Then the reaction mixture was concentrated under reduced pressure to remove the MeOH. The residue was acidified with HCl solution (6 N) to pH 4-5, extracted with EA (1500 mL) for three times. The combined organic layer was washed with sat. brine (1500 mL), dried over Na₂SO₄, and concentrated in vacuo to afford compound B4 (450 g, 40.2% yield). MS: calc'd 177 [(M+H)⁺], measured 177 [(M+H)⁺]; ¹H NMR (400 MHz, DMSO-d₆) δ ppm 11.68 (s, 1H), 8.63 (dd, J=4.60, 1.80 Hz, 1H), 8.22 (dd, J=7.80, 1.80 Hz, 1H), 7.27 (dd, J=7.80, 4.60 Hz, 1H), 5.93 (s, 1H), 3.59 (s, 3H)

### Step (d): Preparation of 4-chloro-1-methyl-1,8-naphthyridin-2-one (Intermediate A5)

A solution of 4-hydroxy-1-methyl-1,8-naphthyridin-2-one (compound B4, 150 g, 0.85 mol) in phosphorus oxychloride (300 mL) was stirred at 100° C. for 2 hrs. The reaction mixture was concentrated in reduced pressure to remove the phosphorus oxychloride. The residue was neutralized by adding saturated aqueous NaHCO₃ at room temperature to pH 7-8, and the mixture was extracted with DCM (1000 mL) twice. The combined organic layer was washed sat. brine (500 mL), dried over Na₂SO₄ and concentrated in vacuo to give a crude product, which was purified by silica gel chromatography (PE/EtOAc=1:0 to 7:1) to afford Intermediate B (39 g, 24% yield). MS: calc'd 195 [(M+H)⁺], measured 195 [(M+H)⁺]; ¹H NMR (400 MHz, DMSO-d₆) δ ppm 8.75 (dd, J=4.60, 1.60 Hz, 1H), 8.32 (dd, J=7.90, 1.70 Hz, 1H), 7.44 (dd, J=8.00, 4.60 Hz, 1H), 7.03 (s, 1H), 3.66 (s, 3H).

### Intermediate C

### 4-Bromo-1-ethyl-1,8-naphthyridin-2-one

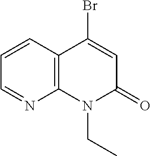

The titled compound was synthesized according to the following scheme:

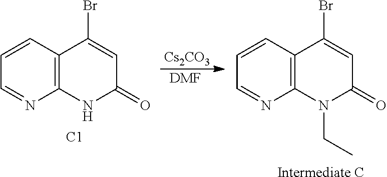

### Step (a): Preparation of 4-bromo-1-ethyl-1,8-naphthyridin-2-one (Intermediate C)

To a solution of 4-bromo-1,8-naphthyridin-2(1H)-one (compound C1, 500 mg, 2.2 mmol) in DMF (20 mL) was added iodoethane (3.47 g, 22.2 mmol) and Cs₂CO₃ (1.45 g, 4.44 mmol). The reaction mixture was stirred at 80° C. overnight. After being cooled to room temperature, the reaction was quenched by adding ice-water (30 mL). The resultant mixture was extracted with PE/EA=1/1 (30 mL) for three times. The combined organic layer was washed sat. brine, dried over Na₂SO₄ and concentrated in vacuo. The residue was purified by flash chromatography (silica gel, 12 g, 10% to 30% EA in PE) to afford Intermediate C (490 mg, 87% yield). MS: calc'd 253 and 255 [(M+H)⁺], measured 253 and 255 [(M+H)⁺].

### Example 1

### 4-[(4R,10bS)-8-(3-amino-3-methyl-azetidin-1-yl)-4-methyl-3,4,6,10b-tetrahydro-1H-pyrazino[2,1-a]isoindol-2-yl]-1-methyl-1,8-naphthyridin-2-one

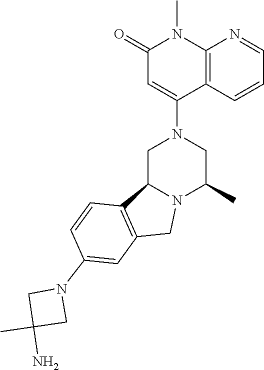 [SMILES: C[C@@H]1CN(c2cc(=O)n(C)c3ncccc23)C[C@@H]2c3ccc(N4CC(C)(N)C4)cc3CN12]

The titled compound was synthesized according to the following scheme:

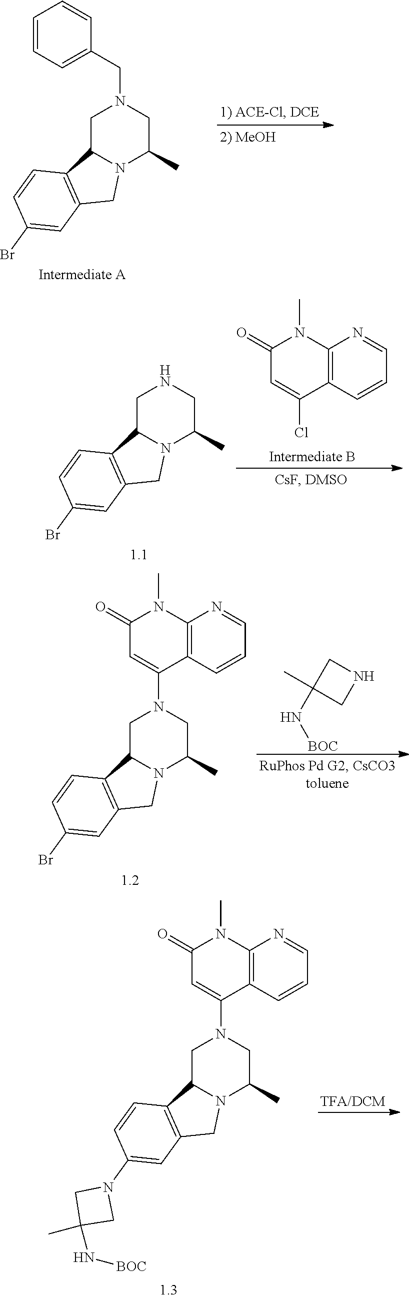

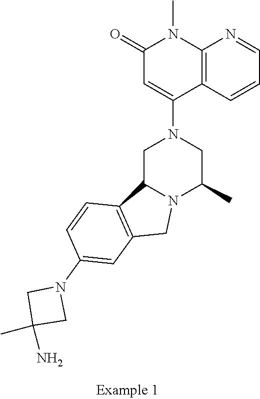

### Step 1: Preparation of (4R,10bS)-8-bromo-4-methyl-1,2,3,4,6,10b-hexahydropyrazino[2,1-a]isoindole (compound 1.1)

To a stirred solution of (4R,10bS)-2-benzyl-8-bromo-4-methyl-3,4,6,10b-tetrahydro-1H-pyrazino[2,1-a]isoindole (Intermediate A, 900 mg, 2.4 mmol) in DCE (30 mL) at room temperature was added 1-chloroethyl carbonochloridate (1.7 g, 12.1 mmol). The reaction mixture was heated under reflux overnight and cooled to room temperature before concentrated in vacuo. The residue was dissolved in MeOH (20 mL) and the resultant mixture was heated under reflux for additional 2 hrs, then concentrated in vacuo. The residue was diluted with water (10 mL), and the solution was basified with aq.NaHCO₃, and the mixture was extracted with EtOAc twice. The combined organic layer was washed with brine, dried over Na₂SO₄, filtered and concentrated in vacuo to afford compound 1.1 (660 mg, 98% yield) which was used directly in the next step. MS: calc'd 267 and 269 [(M+H)⁺], measured 267 and 269 [(M+H)⁺].

### Step 2: Preparation of 1-methyl-4-[(4R,10bS)-8-bromo-4-methyl-3,4,6,10b-tetrahydro-1H-pyrazino [2,1-a] isoindol-2-yl]-1,8-naphthyridin-2-one (compound 1.2)

To a solution of (4R,10bS)-8-bromo-4-methyl-1,2,3,4,6,10b-hexahydropyrazino[2,1-a]isoindole (compound 1.1, 0.70 g, 2.62 mmol) in DMSO (20 mL) was added CsF (1.19 g, 7.86 mmol) and 4-chloro-1-methyl-1,8-naphthyridin-2(1H)-one (Intermediate B, 0.54 g, 2.75 mmol). The reaction mixture was stirred at 120° C. for 20 hrs. After being cooled to room temperature, the reaction was quenched with water (50 mL), and extracted with DCM (50 mL) twice. The combined organic layer was washed with brine, dried over Na₂SO₄, filtered and concentrated in vacuo. The residue was purified by flash chromatography (silica gel, 40 g, 0% to 100% EtOAc in DCM) to afford compound 1.2 (0.63 g, 56.5% yield). MS: calc'd 425 and 427 [(M+H)⁺], measured 425 and 427 [(M+H)⁺].

### Step 3: Preparation of tert-butyl N-[3-methyl-1-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]azetidin-3-yl]carbamate (compound 1.3)

To a solution of 1-methyl-4-[(4R,10bS)-8-bromo-4-methyl-3,4,6,10b-tetrahydro-1H-pyrazino [2,1-a] isoindol-2-yl]-1,8-naphthyridin-2-one (compound 1.2, 60 mg, 141 μmol) in toluene (8 mL) was added tert-butyl (3-methylazetidin-3-yl)carbamate (31.5 mg, 169 μmol), Cs₂CO₃ (138 mg, 423 μmol) and RuPhos Pd G2 (CAS: 1375325-68-0, Aldrich, Catalog: 753246, 21.9 mg, 28.2 μmol). The resultant mixture was heated at 100° C. for 20 hrs. After being cooled to room temperature, diluted with water (50 mL) and extracted with DCM (50 mL) for three times. The combined organic layer was washed with brine, dried over Na₂SO₄, filtered and concentrated in vacuo. The residue was purified by flash chromatography (silica gel, 40 g, 0% to 100% EtOAc in PE) to afford compound 1.3 (40 mg, 53.4% yield). MS: calc'd 531 [(M+H)⁺], measured 531 [(M+H)⁺].

### Step 4: Preparation of 4-[(4R,10bS)-8-(3-amino-3-methyl-azetidin-1-yl)-4-methyl-3,4, 6,10b-tetrahydro-1H-pyrazino[2,1-a]isoindol-2-yl]-1-methyl-1,8-naphthyridin-2-one

### Example 1

To a solution of tert-butyl N-[3-methyl-1-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]azetidin-3-yl]carbamate (compound 1.3, 40 mg, 75.4 μmol) in DCM (5 mL) was added TFA (2 mL). The reaction mixture was stirred at room temperature for 2 hrs, then concentrated to afford a crude product, which was purified by pre-HPLC to afford Example 1 (15 mg, 46.3% yield).). MS: calc'd 431 [(M+H)⁺], measured 431 [(M+H)⁺]. ¹H NMR (400 MHz, METHANOL-d₄) δ ppm 8.75-8.59 (m, 1H), 8.43-8.23 (m, 1H), 7.48-7.37 (m, 1H), 7.32 (d, J=8.3 Hz, 1H), 6.68 (d, J=1.7 Hz, 1H), 6.60-6.48 (m, 1H), 6.28 (s, 1H), 5.15-5.03 (m, 1H), 4.81 (d, J=13.6 Hz, 1H), 4.49 (d, J=13.6 Hz, 1H), 4.26-4.11 (m, 1H), 4.04 (d, J=8.4 Hz, 2H), 3.98-3.86 (m, 3H), 3.78 (s, 3H), 3.65 (br d, J=13.1 Hz, 1H), 3.20-3.08 (m, 1H), 3.04-2.90 (m, 1H), 1.70 (s, 3H), 1.51 (d, J=6.7 Hz, 3H).

### Example 2

### 4-[(4R,10bS)-4-methyl-8-(6-methyl-2,6-diazaspiro[3.3]heptan-2-yl)-3,4,6,10b-tetrahydro-1H-pyrazino[2,1-a]isoindol-2-yl]-1-methyl-1,8-naphthyridin-2-one

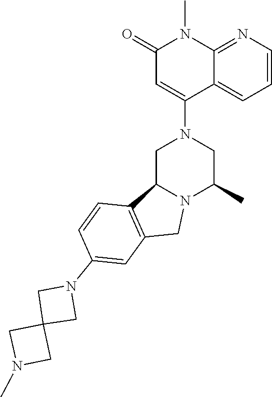 [SMILES: C[C@@H]1CN(c2cc(=O)n(C)c3ncccc23)C[C@@H]2c3ccc(N4CC5(CN(C)C5)C4)cc3CN12]

The title compound was prepared in analogy to the preparation of Example 1 by using 2-methyl-2,6-diazaspiro[3.3]heptane (CAS: 1203567-11-6, PharmaBlock, Catalog: PBLJ2831) instead of tert-butyl N-(3-methylazetidin-3-yl)carbamate in step 3. Example 2 was obtained. MS: calc'd 457 [(M+H)⁺], measured 457 [(M+H)⁺]. ¹H NMR (400 MHz, METHANOL-d₄) δ ppm 8.74-8.62 (m, 1H), 8.39-8.23 (m, 1H), 7.42-7.31 (m, 1H), 7.27 (d, J=8.2 Hz, 1H), 6.60 (d, J=1.7 Hz, 1H), 6.54-6.43 (m, 1H), 6.26 (s, 1H), 5.05-4.95 (m, 1H), 4.76 (d, J=13.6 Hz, 1H), 4.50-4.30 (m, 5H), 4.12-4.08 (m, 1H), 4.08 (s, 4H), 3.93-3.86 (m, 1H), 3.77 (s, 3H), 3.66-3.55 (m, 1H), 3.15-3.05 (m, 1H), 3.01-2.88 (m, 1H), 2.94 (s, 3H), 1.47 (d, J=6.7 Hz, 3H).

### Example 3

### 4-[(4R,10bS)-4-methyl-8-(5-oxa-2,8-diazaspiro[3.5]nonan-2-yl)-3,4,6,10b-tetrahydro-1H-pyrazino[2,1-a]isoindol-2-yl]-1-methyl-1,8-naphthyridin-2-one

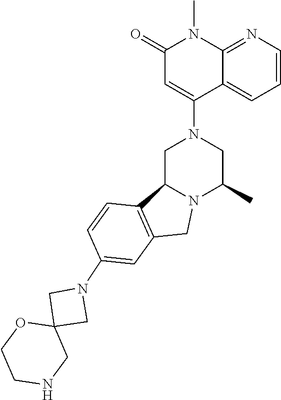 [SMILES: C[C@@H]1CN(c2cc(=O)n(C)c3ncccc23)C[C@@H]2c3ccc(N4CC5(CNCCO5)C4)cc3CN12]

The title compound was prepared in analogy to the preparation of Example 1 by using tert-butyl 5-oxa-2,8-diazaspiro[3.5]nonane-8-carboxylate (CAS: 1251005-61-4, PharmaBlock, Catalog: PBN20111065) instead of tert-butyl N-(3-methylazetidin-3-yl)carbamate in step 3. Example 3 was obtained. MS: calc'd 473 [(M+H)⁺], measured 473 [(M+H)⁺]. ¹H NMR (400 MHz, METHANOL-d₄) δ ppm 8.74-8.65 (m, 1H), 8.44-8.27 (m, 1H), 7.46-7.36 (m, 1H), 7.33 (d, J=8.3 Hz, 1H), 6.67 (d, J=1.7 Hz, 1H), 6.63-6.51 (m, 1H), 6.29 (s, 1H), 5.19-5.08 (m, 1H), 4.85 (d, J=13.7 Hz, 1H), 4.54 (d, J=13.7 Hz, 1H), 4.29-4.14 (m, 1H), 4.08 (d, J=8.6 Hz, 2H), 4.01-3.88 (m, 3H), 3.82 (d, J=8.4 Hz, 2H), 3.78 (s, 3H), 3.66 (br d, J=13.1 Hz, 1H), 3.54 (s, 2H), 3.30-3.24 (m, 2H), 3.21-3.11 (m, 1H), 3.05-2.95 (m, 1H), 1.52 (d, J=6.6 Hz, 3H).

### Example 4

### 4-[(4R,10bS)-8-[(3R)-3-amino-3-methyl-pyrrolidin-1-yl]-4-methyl-3,4,6,10b-tetrahydro-1H-pyrazino[2,1-a]isoindol-2-yl]-1-methyl-1,8-naphthyridin-2-one

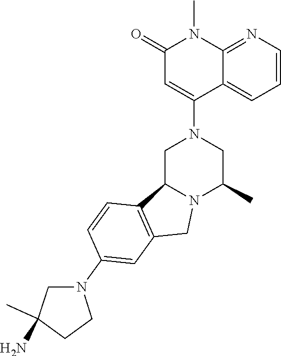 [SMILES: C[C@@H]1CN(c2cc(=O)n(C)c3ncccc23)C[C@@H]2c3ccc(N4CC[C@@](C)(N)C4)cc3CN12]

The title compound was prepared in analogy to the preparation of Example 1 by using tert-butyl N-[(3R)-3-methylpyrrolidin-3-yl]carbamate (CAS: 167888-15-5, PharmaBlock, Catalog: PBXA3113) instead of tert-butyl N-(3-methylazetidin-3-yl)carbamate in step 3. Example 4 was obtained. MS: calc'd 445 [(M+H)⁺], measured 445 [(M+H)⁺]. ¹H NMR (400 MHz, METHANOL-d₄) δ ppm 8.74-8.65 (m, 1H), 8.44-8.35 (m, 1H), 7.47-7.36 (m, 1H), 7.32 (d, J=8.3 Hz, 1H), 6.78 (d, J=1.8 Hz, 1H), 6.72-6.59 (m, 1H), 6.29 (s, 1H), 5.07-4.99 (m, 1H), 4.79 (d, J=13.6 Hz, 1H), 4.45 (d, J=13.4 Hz, 1H), 4.17-4.00 (m, 1H), 3.99-3.87 (m, 1H), 3.79 (s, 3H), 3.71-3.57 (m, 3H), 3.53-3.43 (m, 1H), 3.39 (d, J=10.6 Hz, 1H), 3.19-3.07 (m, 1H), 3.06-2.90 (m, 1H), 2.37-2.20 (m, 2H), 1.60 (s, 3H), 1.50 (d, J=6.7 Hz, 3H).

### Example 5

### 4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-methyl-1,8-naphthyridin-2-one

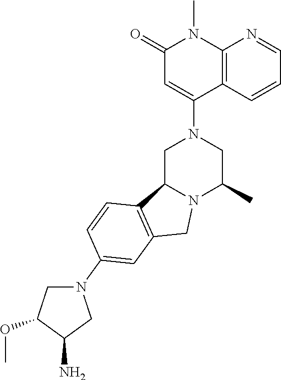 [SMILES: CO[C@@H]1CN(c2ccc3c(c2)CN2[C@H](C)CN(c4cc(=O)n(C)c5ncccc45)C[C@H]32)C[C@H]1N]

The title compound was prepared in analogy to the preparation of Example 1 by using tert-butyl N-[(3R,4R)-4-methoxypyrrolidin-3-yl]carbamate (CAS: 1932066-52-8, PharmaBlock, Catalog: PBZ4728) instead of tert-butyl N-(3-methylazetidin-3-yl)carbamate in step 3. Example 5 was obtained. MS: calc'd 461 [(M+H)⁺], measured 461 [(M+H)⁺]. ¹H NMR (400 MHz, METHANOL-d₄) δ ppm 8.73-8.68 (m, 1H), 8.49-8.28 (m, 1H), 7.43-7.36 (m, 1H), 7.29 (d, J=8.3 Hz, 1H), 6.79 (d, J=1.6 Hz, 1H), 6.68-6.62 (m, 1H), 6.28 (s, 1H), 4.83-4.75 (m, 1H), 4.68 (d, J=13.2 Hz, 1H), 4.28 (d, J=13.2 Hz, 1H), 4.19-4.07 (m, 1H), 3.97-3.83 (m, 4H), 3.80 (s, 3H), 3.76-3.66 (m, 1H), 3.64-3.55 (m, 1H), 3.52-3.41 (m, 2H), 3.49 (s, 3H), 3.09-2.91 (m, 2H), 1.45 (d, J=6.6 Hz, 3H).

### Example 6

### 4-[(4R,10bS)-8-[(4aR,7aR)-3,4,4a,5,7,7a-hexahydro-2H-pyrrolo[3,4-b][1,4]oxazin-6-yl]-4-methyl-3,4,6,10b-tetrahydro-1H-pyrazino[2,1-a]isoindol-2-yl]-1-methyl-1,8-naphthyridin-2-one

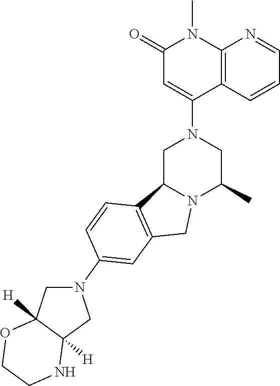 [SMILES: C[C@@H]1CN(c2cc(=O)n(C)c3ncccc23)C[C@@H]2c3ccc(N4C[C@H]5NCCO[C@@H]5C4)cc3CN12]

The title compound was prepared in analogy to the preparation of Example 1 by using tert-butyl (4aR,7aR)-3,4a,5,6,7,7a-hexahydro-2H-pyrrolo[3,4-b][1,4]oxazine-4-carboxylate (CAS:1932337-68-2, PharmaBlock, Catalog: PBXA8123) instead of tert-butyl N-(3-methylazetidin-3-yl)carbamate in step 3. Example 6 was obtained. MS: calc'd 473 [(M+H)⁺], measured 473 [(M+H)⁺]. ¹H NMR (400 MHz, METHANOL-d₄) δ ppm 8.74-8.64 (m, 1H), 8.40-8.31 (m, 1H), 7.40-7.35 (m, 1H), 7.30 (d, J=8.4 Hz, 1H), 6.72 (d, J=1.8 Hz, 1H), 6.63-6.56 (m, 1H), 6.27 (s, 1H), 5.05-4.94 (m, 1H), 4.77 (d, J=13.6 Hz, 1H), 4.42 (d, J=13.4 Hz, 1H), 4.27-4.19 (m, 1H), 4.15-3.84 (m, 4H), 3.81-3.68 (m, 2H), 3.77 (s, 3H), 3.65-3.34 (m, 5H), 3.35-3.32 (m, 1H), 3.13-3.05 (m, 1H), 3.02-2.88 (m, 1H), 1.47 (d, J=6.7 Hz, 3H).

### Example 7

### 4-[(4R,10bS)-8-[(3R,4S)-3-amino-4-fluoro-pyrrolidin-1-yl]-4-methyl-3,4,6,10b-tetrahydro-1H-pyrazino[2,1-a]isoindol-2-yl]-1-methyl-1,8-naphthyridin-2-one

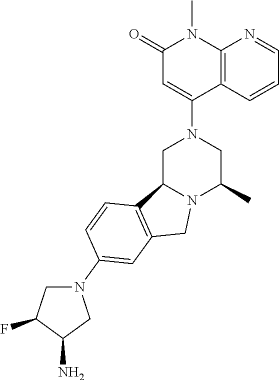 [SMILES: C[C@@H]1CN(c2cc(=O)n(C)c3ncccc23)C[C@@H]2c3ccc(N4C[C@@H](N)[C@@H](F)C4)cc3CN12]

The title compound was prepared in analogy to the preparation of Example 1 by using tert-butyl N-[(3R,4S)-4-fluoropyrrolidin-3-yl]carbamate (CAS: 1033718-91-0, PharmaBlock, Catalog: PB09204) instead of tert-butyl N-(3-methylazetidin-3-yl)carbamate in step 3. Example 7 was obtained. MS: calc'd 449 [(M+H)⁺], measured 449 [(M+H)⁺]. ¹H NMR (400 MHz, METHANOL-d₄) δ ppm 8.74-8.60 (m, 1H), 8.40-8.30 (m, 1H), 7.44-7.37 (m, 1H), 7.34 (d, J=8.3 Hz, 1H), 6.79 (d, J=1.5 Hz, 1H), 6.71-6.59 (m, 1H), 6.29 (s, 1H), 5.62-5.41 (m, 1H), 5.12-5.03 (m, 1H), 4.82 (d, J=13.6 Hz, 1H), 4.49 (d, J=13.6 Hz, 1H), 4.29-4.06 (m, 2H), 3.98-3.61 (m, 5H), 3.78 (s, 3H), 3.49 (t, J=9.1 Hz, 1H), 3.19-3.09 (m, 1H), 3.07-2.93 (m, 1H), 1.51 (d, J=6.6 Hz, 3H).

### Example 8

### 4-[(4R,10bS)-8-[(3S,4S)-4-amino-3-methoxy-1-piperidyl]-4-methyl-3,4,6,10b-tetrahydro-1H-pyrazino[2,1-a]isoindol-2-yl]-1-methyl-1,8-naphthyridin-2-one

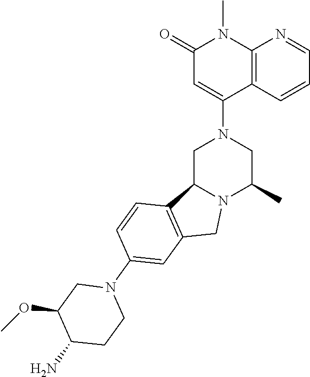 [SMILES: CO[C@H]1CN(c2ccc3c(c2)CN2[C@H](C)CN(c4cc(=O)n(C)c5ncccc45)C[C@H]32)CC[C@@H]1N]

The title compound was prepared in analogy to the preparation of Example 1 by using tert-butyl N-[(3S,4S)-3-methoxy-4-piperidyl]carbamate (CAS: 907544-19-8, PharmaBlock, Catalog: PB07429) instead of tert-butyl N-(3-methylazetidin-3-yl) carbamate in step 3. Example 8 was obtained. MS: calc'd 475 [(M+H)⁺], measured 475 [(M+H)⁺]. ¹H NMR (400 MHz, METHANOL-d₄) δ ppm 8.73-8.66 (m, 1H), 8.46-8.31 (m, 1H), 7.53-7.28 (m, 2H), 7.19 (d, J=2.0 Hz, 1H), 7.15-7.03 (m, 1H), 6.29 (s, 1H), 5.22-5.14 (m, 1H), 4.88 (br d, J=13.7 Hz, 1H), 4.58 (d, J=13.7 Hz, 1H), 4.35-4.11 (m, 2H), 4.01-3.91 (m, 1H), 3.88-3.80 (m, 1H), 3.77 (s, 3H), 3.66 (br d, J=13.1 Hz, 1H), 3.55 (s, 3H), 3.47-3.36 (m, 1H), 3.25-3.09 (m, 2H), 3.08-2.96 (m, 1H), 2.94-2.80 (m, 1H), 2.65-2.53 (m, 1H), 2.25-2.11 (m, 1H), 1.89-1.74 (m, 1H), 1.54 (d, J=6.7 Hz, 3H).

### Example 9

### 4-[(4R,10bS)-8-[(3S,4S)-3-amino-4-methoxy-1-piperidyl]-4-methyl-3,4,6,10b-tetrahydro-1H-pyrazino[2,1-a]isoindol-2-yl]-1-methyl-1,8-naphthyridin-2-one

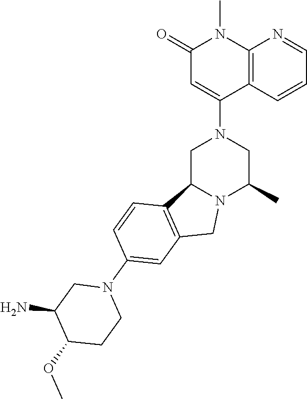 [SMILES: CO[C@H]1CCN(c2ccc3c(c2)CN2[C@H](C)CN(c4cc(=O)n(C)c5ncccc45)C[C@H]32)C[C@@H]1N]

The title compound was prepared in analogy to the preparation of Example 1 by using tert-butyl N-[(3S,4S)-4-methoxy-3-piperidyl]carbamate (PharmaBlock, Catalog: PBZ5290) instead of tert-butyl N-(3-methylazetidin-3-yl) carbamate in step 3. Example 9 was obtained. MS: calc'd 475 [(M+H)⁺], measured 475 [(M+H)⁺]. ¹H NMR (400 MHz, METHANOL-d₄) δ ppm 8.76-8.61 (m, 1H), 8.45-8.24 (m, 1H), 7.46-7.33 (m, 2H), 7.17 (d, J=1.8 Hz, 1H), 7.12-7.01 (m, 1H), 6.29 (s, 1H), 5.26-5.08 (m, 1H), 4.86 (br d, J=13.7 Hz, 1H), 4.55 (d, J=13.7 Hz, 1H), 4.20 (br s, 1H), 4.06-3.88 (m, 2H), 3.84-3.73 (m, 1H), 3.77 (s, 3H), 3.69-3.58 (m, 1H), 3.48-3.39 (m, 1H), 3.47 (s, 3H), 3.29-3.09 (m, 2H), 3.07-2.87 (m, 3H), 2.48-2.29 (m, 1H), 1.67-1.55 (m, 1H), 1.52 (d, J=6.7 Hz, 3H).

### Example 10

### 4-[(4R,10bS)-8-[(2S)-2-(methoxymethyl)piperazin-1-yl]-4-methyl-3,4,6,10b-tetrahydro-1H-pyrazino[2,1-a]isoindol-2-yl]-1-methyl-1,8-naphthyridin-2-one

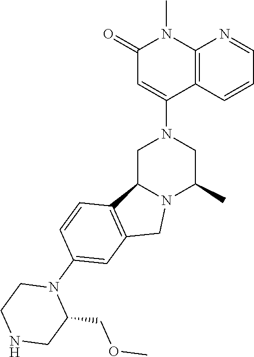 [SMILES: COC[C@@H]1CNCCN1c1ccc2c(c1)CN1[C@H](C)CN(c3cc(=O)n(C)c4ncccc34)C[C@H]21]

The title compound was prepared in analogy to the preparation of Example 1 by using tert-butyl (3S)-3-(methoxymethyl)piperazine-1-carboxylate (CAS: 955400-16-5, Bide Pharmatech, Catalog: BD293888) instead of tert-butyl N-(3-methylazetidin-3-yl) carbamate in step 3. Example 10 was obtained. MS: calc'd 475 [(M+H)⁺], measured 475 [(M+H)⁺]. ¹H NMR (400 MHz, METHANOL-d₄) δ ppm 8.73-8.65 (m, 1H), 8.43-8.29 (m, 1H), 7.41-7.30 (m, 2H), 7.17 (d, J=1.7 Hz, 1H), 7.09-7.00 (m, 1H), 6.28 (s, 1H), 4.98-4.94 (m, 1H), 4.77 (br d, J=13.4 Hz, 1H), 4.40 (br d, J=13.4 Hz, 1H), 4.28-4.17 (m, 1H), 4.10-3.99 (m, 1H), 3.98-3.87 (m, 1H), 3.77 (s, 3H), 3.68-3.41 (m, 8H), 3.30 (s, 3H), 3.28-3.19 (m, 1H), 3.13-2.93 (m, 2H), 1.46 (d, J=6.6 Hz, 3H).

### Example 11

### 4-[(4R,10bS)-4-methyl-8-(5-oxa-2,8-diazaspiro[3.5]nonan-8-yl)-3,4,6,10b-tetrahydro-1H-pyrazino[2,1-a]isoindol-2-yl]-1-methyl-1,8-naphthyridin-2-one

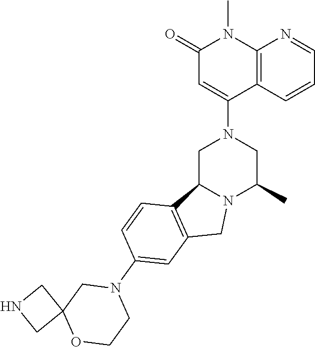 [SMILES: C[C@@H]1CN(c2cc(=O)n(C)c3ncccc23)C[C@@H]2c3ccc(N4CCOC5(CNC5)C4)cc3CN12]

The title compound was prepared in analogy to the preparation of Example 1 by using tert-butyl 5-oxa-2,8-diazaspiro[3.5]nonane-2-carboxylate (CAS: 1251011-05-8, PharmaBlock, Catalog: PBN20111063) instead of tert-butyl N-(3-methylazetidin-3-yl) carbamate in step 3. Example 11 was obtained. MS: calc'd 473 [(M+H)⁺], measured 473 [(M+H)⁺]. ¹H NMR (400 MHz, METHANOL-d₄) δ ppm 8.73-8.64 (m, 1H), 8.40-8.30 (m, 1H), 7.42-7.31 (m, 2H), 7.16 (d, J=1.7 Hz, 1H), 7.11-7.00 (m, 1H), 6.27 (s, 1H), 5.01-4.94 (m, 1H), 4.76 (d, J=13.4 Hz, 1H), 4.40 (d, J=13.4 Hz, 1H), 4.17-4.00 (m, 5H), 3.96-3.90 (m, 1H), 3.90-3.84 (m, 2H), 3.77 (s, 3H), 3.62 (br d, J=12.8 Hz, 1H), 3.40 (s, 2H), 3.17-3.13 (m, 2H), 3.12-2.94 (m, 2H), 1.47 (d, J=6.6 Hz, 3H).

### Example 12

### 4-[(4R,10bS)-4-methyl-8-piperazin-1-yl-3,4,6,10b-tetrahydro-1H-pyrazino[2,1-a]isoindol-2-yl]-1-methyl-1,8-naphthyridin-2-one

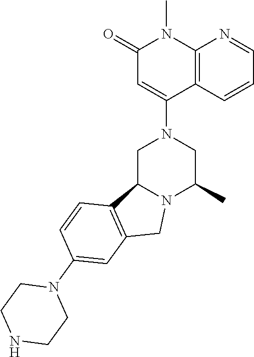 [SMILES: C[C@@H]1CN(c2cc(=O)n(C)c3ncccc23)C[C@@H]2c3ccc(N4CCNCC4)cc3CN12]

The title compound was prepared in analogy to the preparation of Example 1 by using tert-butyl piperazine-1-carboxylate instead of tert-butyl N-(3-methylazetidin-3-yl) carbamate in step 3. Example 12 was obtained. MS: calc'd 431 [(M+H)⁺], measured 431 [(M+H)⁺]. ¹H NMR (400 MHz, METHANOL-d₄) δ ppm 8.73-8.66 (m, 1H), 8.41-8.31 (m, 1H), 7.42-7.37 (m, 1H), 7.31 (d, J=8.3 Hz, 1H), 7.16 (d, J=1.8 Hz, 1H), 7.05-6.98 (m, 1H), 6.27 (s, 1H), 4.57 (br d, J=13.0 Hz, 2H), 4.11 (d, J=12.8 Hz, 1H), 3.99-3.88 (m, 1H), 3.82-3.69 (m, 1H), 3.79 (s, 3H), 3.61-3.54 (m, 1H), 3.49-3.38 (m, 8H), 3.03-2.89 (m, 2H), 1.39 (d, J=6.5 Hz, 3H).

### Example 13

### 4-[(4R,10bS)-4-methyl-8-[(3S)-3-methylpiperazin-1-yl]-3,4,6,10b-tetrahydro-1H-pyrazino[2,1-a]isoindol-2-yl]-1-methyl-1,8-naphthyridin-2-one

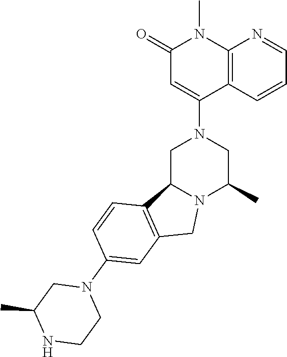 [SMILES: C[C@@H]1CN(c2cc(=O)n(C)c3ncccc23)C[C@@H]2c3ccc(N4CCN[C@@H](C)C4)cc3CN12]

The title compound was prepared in analogy to the preparation of Example 1 by using tert-butyl (2S)-2-methylpiperazine-1-carboxylate instead of tert-butyl N-(3-methylazetidin-3-yl) carbamate in step 3. Example 13 was obtained. MS: calc'd 445 [(M+H)⁺], measured 445 [(M+H)⁺]. ¹H NMR (400 MHz, METHANOL-d₄) δ ppm 8.77-8.62 (m, 1H), 8.40-8.27 (m, 1H), 7.40-7.28 (m, 2H), 7.17 (d, J=1.8 Hz, 1H), 7.08-6.99 (m, 1H), 6.26 (s, 1H), 4.85-4.79 (m, 1H), 4.69 (d, J=13.3 Hz, 1H), 4.30 (d, J=13.2 Hz, 1H), 3.98-3.71 (m, 4H), 3.77 (s, 3H), 3.64-3.56 (m, 1H), 3.54-3.45 (m, 2H), 3.34-3.25 (m, 3H), 3.11-2.93 (m, 3H), 2.89-2.80 (m, 1H), 1.44 (d, J=6.7 Hz, 3H), 1.41 (d, J=6.6 Hz, 3H).

### Example 14

### 4-[(4R,10bS)-4-methyl-8-[(3R)-3-methylpiperazin-1-yl]-3,4,6,10b-tetrahydro-1H-pyrazino[2,1-a]isoindol-2-yl]-1-methyl-1,8-naphthyridin-2-one

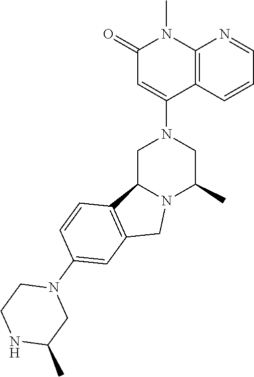 [SMILES: C[C@@H]1CN(c2ccc3c(c2)CN2[C@H](C)CN(c4cc(=O)n(C)c5ncccc45)C[C@H]32)CCN1]

The title compound was prepared in analogy to the preparation of Example 1 by using tert-butyl (2R)-2-methylpiperazine-1-carboxylate instead of tert-butyl N-(3-methylazetidin-3-yl) carbamate in step 3. Example 14 was obtained. MS: calc'd 445 [(M+H)⁺], measured 445 [(M+H)⁺]. ¹H NMR (400 MHz, METHANOL-d₄) δ ppm 8.73-8.65 (m, 1H), 8.41-8.30 (m, 1H), 7.40-7.35 (m, 1H), 7.32 (d, J=8.3 Hz, 1H), 7.17 (d, J=2.0 Hz, 1H), 7.08-6.98 (m, 1H), 6.26 (s, 1H), 4.76-4.68 (m, 1H), 4.64 (d, J=13.2 Hz, 1H), 4.23 (d, J=13.1 Hz, 1H), 3.96-3.90 (m, 1H), 3.89-3.74 (m, 3H), 3.78 (s, 3H), 3.63-3.56 (m, 1H), 3.55-3.42 (m, 2H), 3.34-3.28 (m, 1H), 3.11-2.92 (m, 3H), 2.88-2.74 (m, 1H), 1.42 (d, J=6.7 Hz, 3H), 1.40 (d, J=6.6 Hz, 3H).

### Example 15

### 4-[(4R,10bS)-8-[(3S)-3-amino-3-methyl-1-piperidyl]-4-methyl-3,4,6,10b-tetrahydro-1H-pyrazino[2,1-a]isoindol-2-yl]-1-methyl-1,8-naphthyridin-2-one

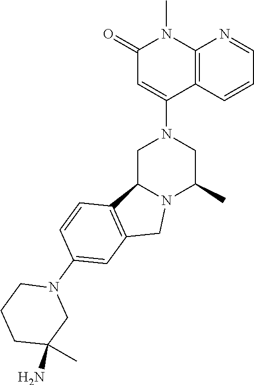 [SMILES: C[C@@H]1CN(c2cc(=O)n(C)c3ncccc23)C[C@@H]2c3ccc(N4CCC[C@](C)(N)C4)cc3CN12]

The title compound was prepared in analogy to the preparation of Example 1 by using tert-butyl N-[(3S)-3-methyl-3-piperidyl]carbamate (CAS: 1363378-21-5, PharmaBlock, Catalog: PBN20120294) instead of tert-butyl N-(3-methylazetidin-3-yl) carbamate in step 3. Example 15 was obtained. MS: calc'd 459 [(M+H)⁺], measured 459 [(M+H)⁺]. ¹H NMR (400 MHz, METHANOL-d₄) δ ppm 8.72-8.64 (m, 1H), 8.40-8.31 (m, 1H), 7.40-7.34 (m, 1H), 7.32 (d, J=8.3 Hz, 1H), 7.19 (d, J=1.8 Hz, 1H), 7.10-6.98 (m, 1H), 6.26 (s, 1H), 4.83-4.77 (m, 1H), 4.68 (d, J=13.2 Hz, 1H), 4.29 (d, J=13.1 Hz, 1H), 3.97-3.86 (m, 2H), 3.78 (s, 3H), 3.65-3.58 (m, 1H), 3.45 (br d, J=12.8 Hz, 2H), 3.06-2.93 (m, 2H), 2.90 (d, J=12.7 Hz, 2H), 2.04-1.77 (m, 3H), 1.72-1.68 (m, 1H), 1.44 (d, J=6.6 Hz, 3H), 1.41 (s, 3H).

### Example 16

### 4-[(4R,10bS)-8-[(3R)-3-amino-1-piperidyl]-4-methyl-3,4,6,10b-tetrahydro-1H-pyrazino[2,1-a]isoindol-2-yl]-1-methyl-1,8-naphthyridin-2-one

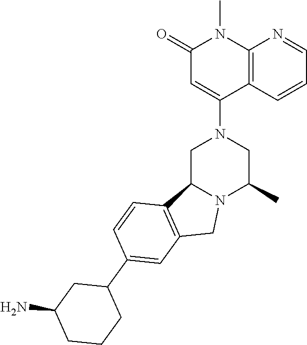 [SMILES: C[C@@H]1CN(c2cc(=O)n(C)c3ncccc23)C[C@@H]2c3ccc(C4CCC[C@@H](N)C4)cc3CN12]

The title compound was prepared in analogy to the preparation of Example 1 by using tert-butyl N-[(3R)-3-piperidyl]carbamate (CAS: 309956-78-3, PharmaBlock, Catalog: PB00803) instead of tert-butyl N-(3-methylazetidin-3-yl) carbamate in step 3. Example 16 was obtained. MS: calc'd 445 [(M+H)⁺], measured 445[(M+H)⁺]. ¹H NMR (400 MHz, METHANOL-d₄) δ ppm 8.70-8.64 (m, 1H), 8.40-8.30 (m, 1H), 7.47-7.32 (m, 2H), 7.16 (d, J=1.8 Hz, 1H), 7.08-6.99 (m, 1H), 6.28 (s, 1H), 5.19-5.07 (m, 1H), 4.84 (d, J=13.7 Hz, 1H), 4.53 (d, J=13.7 Hz, 1H), 4.25-4.09 (m, 1H), 3.99-3.88 (m, 1H), 3.76 (s, 3H), 3.69-3.60 (m, 1H), 3.60-3.53 (m, 1H), 3.52-3.42 (m, 1H), 3.34 (br d, J=3.3 Hz, 1H), 3.20-3.07 (m, 3H), 3.05-2.93 (m, 1H), 2.10-1.90 (m, 2H), 1.84-1.67 (m, 2H), 1.51 (d, J=6.7 Hz, 3H).

### Example 17

### 4-[(4R,10bS)-4-methyl-8-[(2R)-2-methylpiperazin-1-yl]-3,4,6,10b-tetrahydro-1H-pyrazino[2,1-a]isoindol-2-yl]-1-methyl-1,8-naphthyridin-2-one

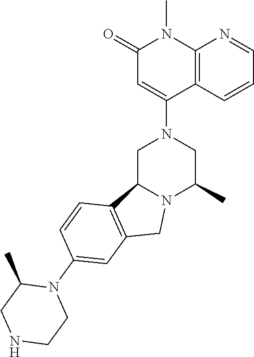 [SMILES: C[C@@H]1CNCCN1c1ccc2c(c1)CN1[C@H](C)CN(c3cc(=O)n(C)c4ncccc34)C[C@H]21]

The title compound was prepared in analogy to the preparation of Example 1 by using tert-butyl (3R)-3-methylpiperazine-1-carboxylate (CAS: 163765-44-4, PharmaBlock, Catalog: PB07855) instead of tert-butyl N-(3-methylazetidin-3-yl) carbamate in step 3. Example 17 was obtained. MS: calc'd 445 [(M+H)⁺], measured 445 [(M+H)⁺]. ¹H NMR (400 MHz, METHANOL-d₄) δ ppm 8.75-8.64 (m, 1H), 8.39-8.33 (m, 1H), 7.43-7.32 (m, 2H), 7.21 (d, J=1.6 Hz, 1H), 7.14-7.04 (m, 1H), 6.28 (s, 1H), 5.08-5.01 (m, 1H), 4.83 (d, J=13.6 Hz, 1H), 4.47 (d, J=13.6 Hz, 1H), 4.17-4.00 (m, 2H), 3.99-3.90 (m, 1H), 3.77 (s, 3H), 3.65 (br d, J=13.0 Hz, 1H), 3.48-3.36 (m, 3H), 3.29-3.18 (m, 3H), 3.15-2.97 (m, 2H), 1.48 (d, J=6.7 Hz, 3H), 1.10 (d, J=6.6 Hz, 3H).

### Example 18

### 4-[(4R,10bS)-8-(4-amino-4-methyl-1-piperidyl)-4-methyl-3,4,6,10b-tetrahydro-1H-pyrazino[2,1-a]isoindol-2-yl]-1-methyl-1,8-naphthyridin-2-one

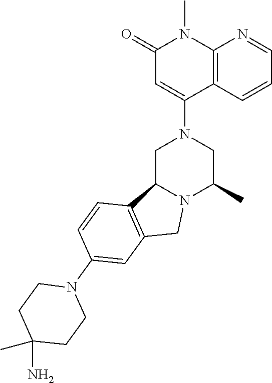 [SMILES: C[C@@H]1CN(c2cc(=O)n(C)c3ncccc23)C[C@@H]2c3ccc(N4CCC(C)(N)CC4)cc3CN12]

The title compound was prepared in analogy to the preparation of Example 1 by using tert-butyl N-(4-methyl-4-piperidyl)carbamate instead of tert-butyl N-(3-methylazetidin-3-yl) carbamate in step 3. Example 18 was obtained. MS: calc'd 459 [(M+H)⁺], measured 459 [(M+H)⁺]. ¹H NMR (400 MHz, METHANOL-d₄) δ ppm 8.70-8.65 (m, 1H), 8.40-8.29 (m, 1H), 7.47-7.29 (m, 2H), 7.15 (d, J=1.8 Hz, 1H), 7.07-6.99 (m, 1H), 6.28 (s, 1H), 5.27-5.12 (m, 1H), 4.90 (d, J=13.7 Hz, 1H), 4.59 (d, J=13.7 Hz, 1H), 4.33-4.17 (m, 1H), 4.11-3.83 (m, 1H), 3.76 (s, 3H), 3.69-3.50 (m, 3H), 3.23-3.09 (m, 3H), 3.09-2.94 (m, 1H), 2.01-1.85 (m, 4H), 1.52 (d, J=6.7 Hz, 3H), 1.46 (s, 3H).

### Example 19

### 4-[(4R,10bS)-8-[(1S,4S)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one

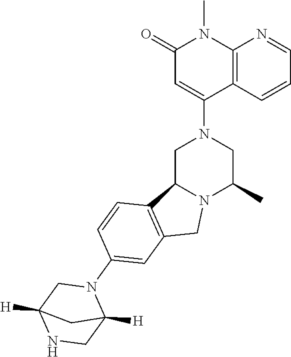 [SMILES: C[C@@H]1CN(c2cc(=O)n(C)c3ncccc23)C[C@@H]2c3ccc(N4C[C@@H]5C[C@H]4CN5)cc3CN12]

The title compound was prepared in analogy to the preparation of Example 1 by using tert-butyl (1S,4S)-2,5-diazabicyclo[2.2.1]heptane-2-carboxylate (CAS: 113451-59-5, PharmaBlock, Catalog: PBN20120579) instead of tert-butyl N-(3-methylazetidin-3-yl) carbamate in step 3. Example 19 was obtained. MS: calc'd 443 [(M+H)⁺], measured 443 [(M+H)⁺]. ¹H NMR (400 MHz, METHANOL-d₄) δ ppm 8.71-8.63 (m, 1H), 8.38-8.32 (m, 1H), 7.42-7.30 (m, 2H), 6.83 (s, 1H), 6.76-6.64 (m, 1H), 6.27 (s, 1H), 5.16-5.06 (m, 1H), 4.86-4.68 (m, 2H), 4.60-4.49 (m, 2H), 4.27-4.11 (m, 1H), 3.92 (br d, J=11.2 Hz, 1H), 3.79-3.72 (m, 1H), 3.75 (s, 3H), 3.64 (br d, J=13.2 Hz, 1H), 3.43-3.33 (m, 3H), 3.23-3.11 (m, 1H), 3.06-2.92 (m, 1H), 2.29 (br d, J=11.1 Hz, 1H), 2.09 (br d, J=11.1 Hz, 1H), 1.51 (d, J=6.7 Hz, 3H).

### Example 20

### 4-[(4R,10bS)-8-[(1R,4R)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one

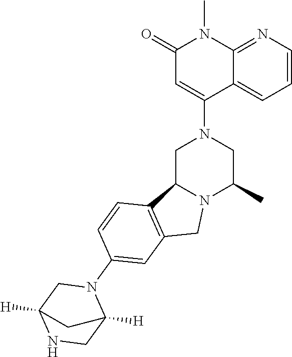 [SMILES: C[C@@H]1CN(c2cc(=O)n(C)c3ncccc23)C[C@@H]2c3ccc(N4C[C@H]5C[C@@H]4CN5)cc3CN12]

The title compound was prepared in analogy to the preparation of Example 1 by using tert-butyl (1R,4R)-2,5-diazabicyclo[2.2.1]heptane-2-carboxylate (CAS: 134003-84-2, PharmaBlock, Catalog: PBN20120578) instead of tert-butyl N-(3-methylazetidin-3-yl) carbamate in step 3. Example 20 was obtained. MS: calc'd 443 [(M+H)⁺], measured 443 [(M+H)⁺]. ¹H NMR (400 MHz, METHANOL-d₄) δ ppm 8.74-8.63 (m, 1H), 8.42-8.29 (m, 1H), 7.43-7.32 (m, 2H), 6.83 (d, J=1.8 Hz, 1H), 6.75-6.62 (m, 1H), 6.27 (s, 1H), 5.23-5.03 (m, 1H), 4.84-4.69 (m, 2H), 4.61-4.47 (m, 2H), 4.31-4.09 (m, 1H), 3.92 (br d, J=12.7 Hz, 1H), 3.82-3.70 (m, 1H), 3.76 (s, 3H), 3.65 (br d, J=13.1 Hz, 1H), 3.45-3.33 (m, 3H), 3.23-3.09 (m, 1H), 3.07-2.89 (m, 1H), 2.29 (br d, J=11.1 Hz, 1H), 2.09 (br d, J=11.1 Hz, 1H), 1.51 (d, J=6.7 Hz, 3H).

### Example 21

### 4-[(4R,10bS)-8-(3,8-diazabicyclo[3.2.1]octan-3-yl)-4-methyl-3,4,6,10b-tetrahydro-1H-pyrazino[2,1-a]isoindol-2-yl]-1-methyl-1,8-naphthyridin-2-one

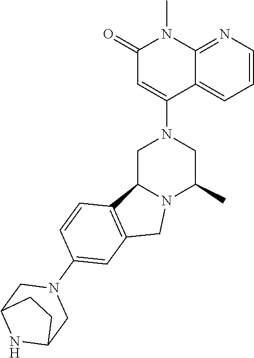 [SMILES: C[C@@H]1CN(c2cc(=O)n(C)c3ncccc23)C[C@@H]2c3ccc(N4CC5CCC(C4)N5)cc3CN12]

The title compound was prepared in analogy to the preparation of Example 1 by using tert-butyl 3,8-diazabicyclo[3.2.1]octane-8-carboxylate (CAS: 149771-44-8, PharmaBlock, Catalog: PBN20120001) instead of tert-butyl N-(3-methylazetidin-3-yl) carbamate in step 3. Example 21 was obtained. MS: calc'd 457 [(M+H)⁺], measured 457 [(M+H)⁺]. ¹H NMR (400 MHz, METHANOL-d₄) δ ppm 8.59-8.53 (m, 1H), 8.27-8.18 (m, 1H), 7.30-7.22 (m, 1H), 7.18 (d, J=8.4 Hz, 1H), 6.97 (d, J=1.8 Hz, 1H), 6.88-6.77 (m, 1H), 6.14 (s, 1H), 4.63-4.56 (m, 1H), 4.51 (d, J=13.2 Hz, 1H), 4.09 (br t, J=6.3 Hz, 3H), 3.84-3.76 (m, 1H), 3.75-3.69 (m, 1H), 3.66 (s, 3H), 3.65-3.58 (m, 2H), 3.46 (br d, J=12.6 Hz, 1H), 3.04 (d, J=12.0 Hz, 2H), 2.92-2.76 (m, 2H), 2.03 (s, 4H), 1.30 (d, J=6.6 Hz, 3H).

### Example 22

### 4-[(4R,10bS)-8-[(6R)-6-amino-1,4-oxazepan-4-yl]-4-methyl-3,4,6,10b-tetrahydro-1H-pyrazino[2,1-a]isoindol-2-yl]-1-methyl-1,8-naphthyridin-2-one

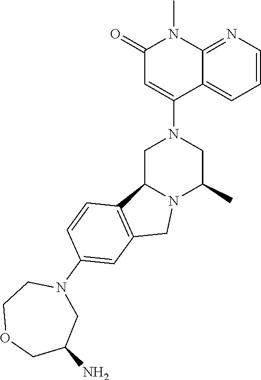 [SMILES: C[C@@H]1CN(c2cc(=O)n(C)c3ncccc23)C[C@@H]2c3ccc(N4CCOC[C@H](N)C4)cc3CN12]

The title compound was prepared in analogy to the preparation of Example 1 by using tert-butyl N-[(6R)-1,4-oxazepan-6-yl]carbamate (PharmaBlock, Catalog: PB97932) instead of tert-butyl N-(3-methylazetidin-3-yl) carbamate in step 3. Example 22 was obtained. MS: calc'd 461 [(M+H)⁺], measured 461 [(M+H)⁺]. ¹H NMR (400 MHz, METHANOL-d₄) δ ppm 8.75-8.67 (m, 1H), 8.43-8.32 (m, 1H), 7.44-7.37 (m, 1H), 7.35 (d, J=8.6 Hz, 1H), 7.03 (d, J=2.0 Hz, 1H), 6.95-6.84 (m, 1H), 6.29 (s, 1H), 5.19-5.07 (m, 1H), 4.85 (d, J=13.7 Hz, 1H), 4.54 (d, J=13.6 Hz, 1H), 4.29-4.12 (m, 2H), 4.09-3.74 (m, 6H), 3.79 (s, 3H), 3.70-3.47 (m, 4H), 3.22-3.10 (m, 1H), 3.08-2.96 (m, 1H), 1.52 (d, J=6.7 Hz, 3H).

### Example 23

### 4-[(4R,10bS)-8-[2-(dimethylamino)ethoxy]-4-methyl-3,4,6,10b-tetrahydro-1H-pyrazino[2,1-a]isoindol-2-yl]-1-methyl-1,8-naphthyridin-2-one

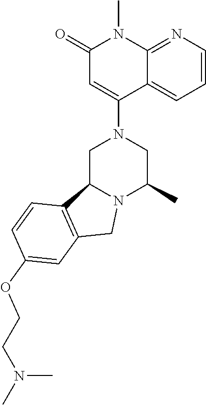 [SMILES: C[C@@H]1CN(c2cc(=O)n(C)c3ncccc23)C[C@@H]2c3ccc(OCCN(C)C)cc3CN12]

The title compound was prepared in analogy to the preparation of Example 1 by using 2-(dimethylamino)ethanol instead of tert-butyl N-(3-methylazetidin-3-yl) carbamate in step 3. Example 23 was obtained. MS: calc'd 434 [(M+H)⁺], measured 434 [(M+H)⁺]. ¹H NMR (400 MHz, METHANOL-d₄) δ ppm 8.74-8.63 (m, 1H), 8.37-8.28 (m, 1H), 7.40-7.33 (m, 1H), 7.28 (d, J=8.3 Hz, 1H), 7.11 (d, J=2.2 Hz, 1H), 7.00-6.92 (m, 1H), 6.25 (s, 1H), 4.43 (d, J=12.7 Hz, 1H), 4.39-4.32 (m, 2H), 4.30 (br d, J=9.5 Hz, 1H), 3.96-3.86 (m, 2H), 3.79 (s, 3H), 3.65-3.58 (m, 2H), 3.56-3.42 (m, 2H), 3.00 (s, 6H), 2.96-2.86 (m, 1H), 2.85-2.75 (m, 1H), 1.32 (d, J=6.4 Hz, 3H).

### Example 24

### 4-[(4R,10bS)-4-methyl-8-(4-piperidyl)-3,4,6,10b-tetrahydro-1H-pyrazino[2,1-a]isoindol-2-yl]-1-methyl-1,8-naphthyridin-2-one

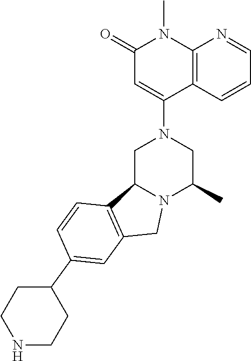 [SMILES: C[C@@H]1CN(c2cc(=O)n(C)c3ncccc23)C[C@@H]2c3ccc(C4CCNCC4)cc3CN12]

The titled compound was synthesized according to the following scheme:

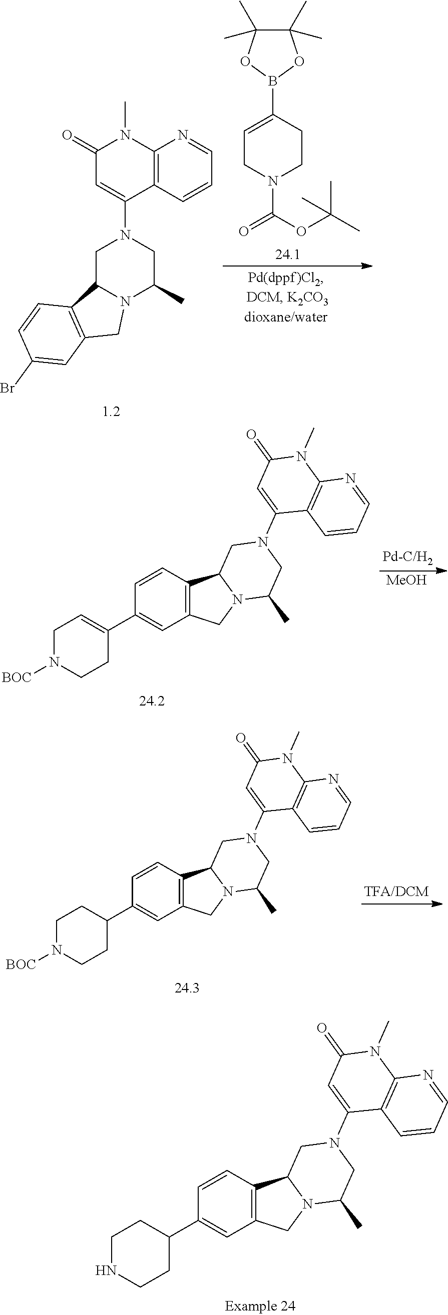

### Step 1: Preparation of tert-butyl 4-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]-3,6-dihydro-2H-pyridine-1-carboxylate (compound 24.2)

To a solution of 4-[(4R,10bS)-8-bromo-4-methyl-3,4,6,10b-tetrahydro-1H-pyrazino[2,1-a]isoindol-2-yl]-1-methyl-1,8-naphthyridin-2-one (compound 1.2, 300 mg, 705 μmol) in dioxane (18 mL) and water (2 mL) was added tert-butyl 4-(4,4,5,5-tetramethyl-1,3,2-dioxaborolan-2-yl)-3,6-dihydro-2H-pyridine-1-carboxylate (compound 24.1, 262 mg, 846 μmol), K₂CO₃ (195 mg, 1.41 mmol) and Pd (dppf) C₁₂-DCM adduct (51.6 mg, 70.5 μmol). The resultant mixture was heated at 100° C. for 20 hrs. After being cooled to room temperature, the reaction mixture was diluted with water (30 mL) and extracted with DCM (60 mL) twice. The combined organic layer was washed with brine, dried over Na₂SO₄, filtered and concentrated in vacuo. The residue was purified by flash chromatography (silica gel, 40 g, 0% to 100% EtOAc in DCM) to afford compound 24.2 (240 mg, 64.5% yield). MS: calc'd 528 [(M+H)⁺], measured 528 [(M+H)⁺].

### Step 2: Preparation of tert-butyl 4-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]piperidine-1-carboxylate (compound 24.3)

A mixture of tert-butyl 4-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]-3,6-dihydro-2H-pyridine-1-carboxylate (compound 24.2, 240 mg, 455 μmol) and Pd—C (30 mg) in MeOH (50 mL) was hydrogenated by a hydrogen balloon at room temperature for 30 min. After the catalyst was filtered off, the filtrate was concentrated in vacuo. The crude material was purified by flash chromatography (silica gel, 40 g, 50% to 100% EtOAc in DCM) to afford compound 24.3 (180 mg, 74.7% yield). MS: calc'd 530 [(M+H)⁺], measured 530 [(M+H)⁺].

### Step 3: Preparation of 4-[(4R,10bS)-4-methyl-8-(4-piperidyl)-3,4,6,10b-tetrahydro-1H-pyrazino[2,1-a]isoindol-2-yl]-1-methyl-1,8-naphthyridin-2-one (Example 24)

To a solution of tert-butyl 4-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]piperidine-1-carboxylate (compound 24.3, 180 mg, 340 μmol) in DCM (20 mL) was added TFA (5 mL). The reaction mixture was stirred at room temperature for 2 hrs, then concentrated to afford a crude product, which was purified by pre-HPLC to afford Example 24 (101 mg, 69.2% yield). MS: calc'd 430 [(M+H)⁺], measured 430 [(M+H)⁺]. ¹H NMR (400 MHz, METHANOL-d₄) δ ppm 8.72-8.63 (m, 1H), 8.39-8.29 (m, 1H), 7.42-7.34 (m, 3H), 7.31-7.23 (m, 1H), 6.26 (s, 1H), 4.78-4.71 (m, 1H), 4.66 (d, J=13.2 Hz, 1H), 4.24 (d, J=13.2 Hz, 1H), 3.99-3.91 (m, 1H), 3.88-3.82 (m, 1H), 3.77 (s, 3H), 3.59 (br d, J=12.7 Hz, 1H), 3.51 (br d, J=12.6 Hz, 2H), 3.23-3.09 (m, 2H), 3.06-2.89 (m, 3H), 2.12-2.03 (m, 2H), 2.00-1.83 (m, 2H), 1.41 (d, J=6.6 Hz, 3H).

### Example 25

### 4-[(4R,10bS)-4-methyl-8-(3-piperidyl)-3,4,6,10b-tetrahydro-1H-pyrazino[2,1-a]isoindol-2-yl]-1-methyl-1,8-naphthyridin-2-one

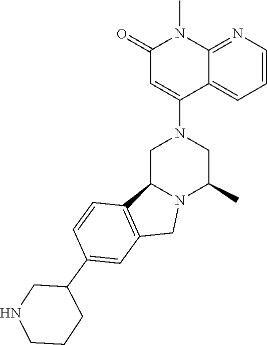 [SMILES: C[C@@H]1CN(c2cc(=O)n(C)c3ncccc23)C[C@@H]2c3ccc(C4CCCNC4)cc3CN12]

The title compound was prepared in analogy to the preparation of Example 24 by using tert-butyl 5-(4,4,5,5-tetramethyl-1,3,2-dioxaborolan-2-yl)-3,6-dihydro-2H-pyridine-1-carboxylate instead of tert-butyl 4-(4,4,5,5-tetramethyl-1,3,2-dioxaborolan-2-yl)-3,6-dihydro-2H-pyridine-1-carboxylate carbamate in step 1. Example 25 was obtained. MS: calc'd 430 [(M+H)⁺], measured 430 [(M+H)⁺]. ¹H NMR (400 MHz, METHANOL-d₄) δ ppm 8.70-8.65 (m, 1H), 8.38-8.33 (m, 1H), 7.47-7.41 (m, 2H), 7.40-7.30 (m, 2H), 6.27 (s, 1H), 4.78-4.71 (m, 1H), 4.74 (d, J=13.3 Hz, 1H), 4.36 (d, J=13.3 Hz, 1H), 4.01-3.92 (m, 2H), 3.77 (s, 3H), 3.66-3.57 (m, 1H), 3.48-3.36 (m, 2H), 3.15-2.96 (m, 5H), 2.11-1.98 (m, 2H), 1.95-1.78 (m, 2H), 1.45 (d, J=6.6 Hz, 3H).

### Example 26

### 4-[(4R,10bS)-4-methyl-8-pyrrolidin-3-yl-3,4,6,10b-tetrahydro-1H-pyrazino[2,1-a]isoindol-2-yl]-1-methyl-1,8-naphthyridin-2-one

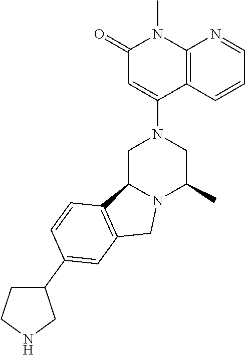 [SMILES: C[C@@H]1CN(c2cc(=O)n(C)c3ncccc23)C[C@@H]2c3ccc(C4CCNC4)cc3CN12]

The title compound was prepared in analogy to the preparation of Example 24 by using tert-butyl 3-(4,4,5,5-tetramethyl-1,3,2-dioxaborolan-2-yl)-2,5-dihydropyrrole-1-carboxylate instead of tert-butyl 4-(4,4,5,5-tetramethyl-1,3,2-dioxaborolan-2-yl)-3,6- dihydro-2H-pyridine-1- carboxylate carbamate in step 1. Example 26 was obtained. MS: calc'd 416 [(M+H)⁺], measured 416 [(M+H)⁺]. ¹H NMR (400 MHz, METHANOL-d₄) δ ppm 8.70-8.63 (m, 1H), 8.38-8.30 (m, 1H), 7.43-7.28 (m, 4H), 6.25 (s, 1H), 4.50 (d, J=12.8 Hz, 1H), 4.45 (br d, J=10.0 Hz, 1H), 4.01 (br d, J=12.5 Hz, 1H), 3.97-3.86 (m, 1H), 3.78 (s, 3H), 3.75-3.67 (m, 1H), 3.63-3.52 (m, 4H), 3.44-3.33 (m, 1H), 3.25-3.16 (m, 1H), 2.99-2.91 (m, 1H), 2.91-2.78 (m, 1H), 2.46 (br d, J=3.3 Hz, 1H), 2.18-2.03 (m, 1H), 1.34 (d, J=6.4 Hz, 3H).

### Example 27A and Example 27B

### 4-[(4R,10bS)-4-methyl-8-(trans-4-morpholinocyclohexyl)-3,4,6,10b-tetrahydro-1H-pyrazino[2,1-a]isoindol-2-yl]-1-methyl-1,8-naphthyridin-2-one and 4-[(4R,10bS)-4-methyl-8-(cis-4-morpholinocyclohexyl)-3,4,6,10b-tetrahydro-1H-pyrazino[2,1-a]isoindol-2-yl]-1-methyl-1,8-naphthyridin-2-one

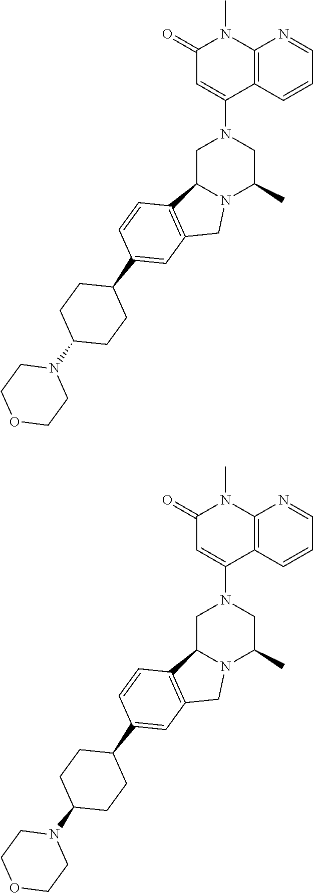 [SMILES: C[C@@H]1CN(c2cc(=O)n(C)c3ncccc23)C[C@@H]2c3ccc([C@H]4CC[C@H](N5CCOCC5)CC4)cc3CN12 ; C[C@@H]1CN(c2cc(=O)n(C)c3ncccc23)C[C@@H]2c3ccc([C@H]4CC[C@@H](N5CCOCC5)CC4)cc3CN12]

The titled compound was synthesized according to the following scheme:

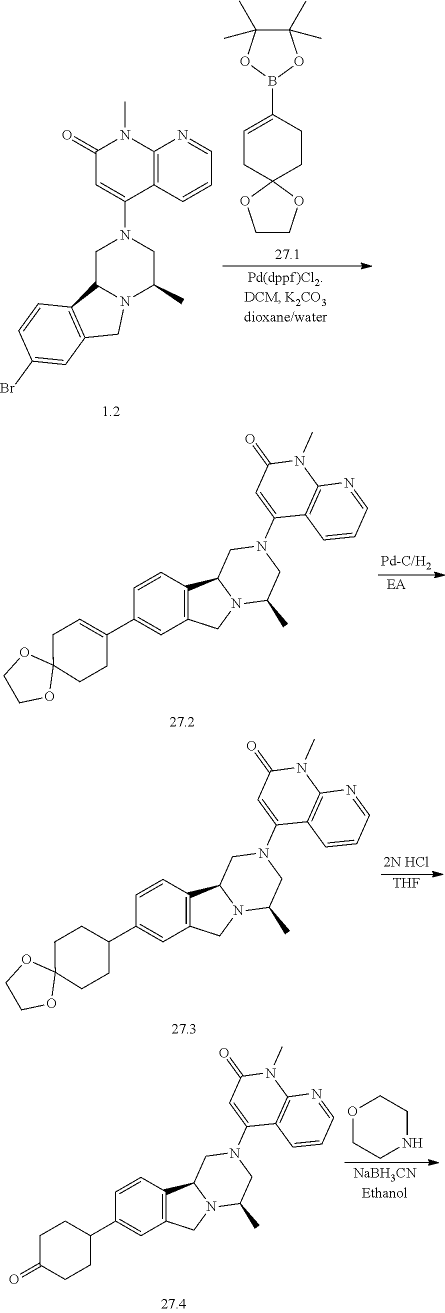

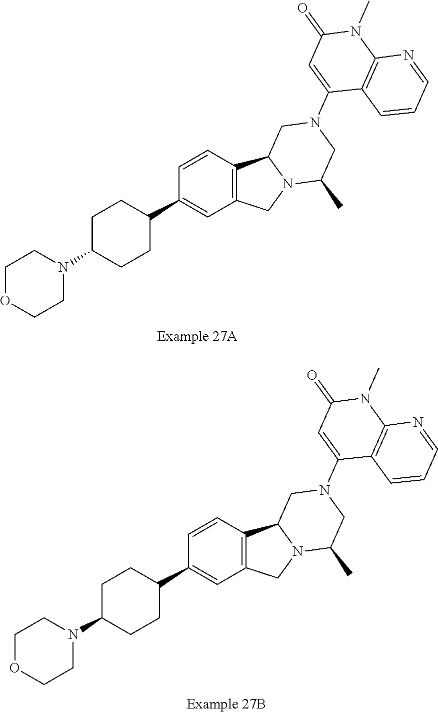

### Step 1: Preparation of 4-[(4R,10bS)-8-(1,4-dioxaspiro[4.5]dec-7-en-8-yl)-4-methyl-3,4,6,10b-tetrahydro-1H-pyrazino[2,1-a]isoindol-2-yl]-1-methyl-1,8-naphthyridin-2-one (compound 27.2)

To a solution of 4-[(4R,10bS)-8-bromo-4-methyl-3,4,6,10b-tetrahydro-1H-pyrazino[2,1-a]isoindol-2-yl]-1-methyl-1,8-naphthyridin-2-one (compound 1.2, 100 mg, 235 μmol) in dioxane (9 mL) and water (1 mL) was added 2-(1,4-dioxaspiro[4.5]dec-7-en-8-yl)-4,4,5,5-tetramethyl-1,3,2-dioxaborolane (compound 27.1, 62.6 mg, 235 μmol), K₂CO₃ (65 mg, 470 μmol) and PdCl₂(dppf). DCM adduct (17.2 mg, 23.5 μmol). The resultant mixture was heated at 100° C. for 20 hrs. After being cooled to room temperature, the reaction mixture was diluted with water (30 mL) and extracted with DCM (60 mL) twice. The combined organic layer was washed with brine, dried over Na₂SO₄, filtered and concentrated in vacuo. The residue was purified by flash chromatography (silica gel, 20 g, 0% to 100% EtOAc in DCM) to afford compound 27.2 (101 mg, 88.6% yield). MS: calc'd 485 [(M+H)⁺], measured 485 [(M+H)⁺].

### Step 2: Preparation of 4-[(4R,10bS)-8-(1,4-dioxaspiro[4.5]decan-8-yl)-4-methyl-3,4,6, 10b-tetrahydro-1H-pyrazino[2,1-a]isoindol-2-yl]-1-methyl-1,8-naphthyridin-2-one (compound 27.3)

A mixture of 4-[(4R,10bS)-8-(1,4-dioxaspiro[4.5]dec-7-en-8-yl)-4-methyl-3,4,6,10b-tetrahydro-1H-pyrazino[2,1-a]isoindol-2-yl]-1-methyl-1,8-naphthyridin-2-one (compound 27.2, 101 mg, 208 μmol) and Pd—C (20 mg) in Ethyl acetate (30 mL) was hydrogenated by a hydrogen balloon at room temperature for 2 hrs. After the catalyst was filtered off, the filtrate was concentrated in vacuo to afford compound 27.3 (90 mg, 88.7% yield) which was used directly for the next step without further purification. MS: calc'd 487 [(M+H)⁺], measured 487 [(M+H)⁺].

### Step 3: Preparation of 4-[(4R,10bS)-4-methyl-8-(4-oxocyclohexyl)-3,4,6,10b-tetrahydro-1H-pyrazino[2,1-a]isoindol-2-yl]-1-methyl-1,8-naphthyridin-2-one (compound 27.4)

To a solution of 4-[(4R,10bS)-8-(1,4-dioxaspiro[4.5]decan-8-yl)-4-methyl-3,4,6,10b-tetrahydro-1H-pyrazino[2,1-a]isoindol-2-yl]-1-methyl-1,8-naphthyridin-2-one (compound 27.3, 90 mg, 185 μmol) in THF (10 mL) was added 2N HCl solution (aq, 2 mL, 4 mmol). The resultant mixture was stirred at reflux for 30 min. After being cooled to room temperature, the reaction mixture was basified with 2N NaOH solution (aq) to pH 8, and extracted with EtOAc twice. The combined organic layer was dried over MgSO₄, filtered and concentrated in vacuo to afford compound 27.4 (70 mg, 158 μmol, 85.5% yield) which was used directly for the next step without further purification. MS: calc'd 443 [(M+H)⁺], measured 443 [(M+H)⁺].

### Step 4: Preparation of 4-[(4R,10bS)-4-methyl-8-(trans-4-morpholinocyclohexyl)-3,4,6,10b-tetrahydro-1H-pyrazino[2,1-a]isoindol-2-yl]-1-methyl-1,8-naphthyridin-2-one and 4-[(4R,10bS)-4-methyl-8-(cis-4-morpholinocyclohexyl)-3,4,6,10b-tetrahydro-1H-pyrazino[2,1-a]isoindol-2-yl]-1-methyl-1,8-naphthyridin-2-one (Example 27A and Example 27B)

The mixture of 4-[(4R,10bS)-4-methyl-8-(4-oxocyclohexyl)-3,4,6,10b-tetrahydro-1H-pyrazino[2,1-a]isoindol-2-yl]-1-methyl-1,8-naphthyridin-2-one (compound 27.4, 70 mg, 158 μmol), morpholine (68.9 mg, 791 μmol) and NaBH₃CN (19.9 mg, 316 μmol) in Ethanol (5 mL) was stirred at reflux for 2 hrs. The mixture was concentrated, the residue was purified by pre-HPLC to afford Example 27A (7.3 mg, 9% yield) and Example 27B (3.6 mg, 4.4% yield), which stereochemistry was determined by NOESY.

Example 27A MS: calc'd 514 [(M+H)⁺], measured 514 [(M+H)⁺]. ¹H NMR (400 MHz, METHANOL-d₄) δ ppm 8.65 (dd, J=1.8, 4.6 Hz, 1H), 8.31 (dd, J=1.8, 8.0 Hz, 1H), 7.34 (dd, J=4.6, 8.1 Hz, 1H), 7.24 (s, 1H), 7.2-7.1 (m, 1H), 7.1-7.1 (m, 1H), 6.22 (s, 1H), 4.26 (d, J=12.2 Hz, 1H), 4.02 (br d, J=10.5 Hz, 1H), 3.89 (br d, J=11.6 Hz, 1H), 3.77 (s, 3H), 3.7-3.7 (m, 4H), 3.7-3.6 (m, 1H), 3.5-3.4 (m, 1H), 3.26 (ddd, J=3.0, 6.8, 10.0 Hz, 1H), 2.88 (t, J=11.0 Hz, 1H), 2.71 (dd, J=10.5, 12.0 Hz, 1H), 2.7-2.6 (m, 4H), 2.6-2.5 (m, 1H), 2.4-2.3 (m, 1H), 2.09 (br d, J=11.1 Hz, 2H), 1.94 (br d, J=12.6 Hz, 2H), 1.6-1.5 (m, 2H), 1.5-1.4 (m, 2H), 1.24 (d, J=6.4 Hz, 3H).

Example 27B MS: calc'd 514 [(M+H)⁺], measured 514 [(M+H)⁺]. ¹H NMR (400 MHz, METHANOL-d₄) δ ppm 8.65 (dd, J=1.8, 4.6 Hz, 1H), 8.31 (dd, J=1.7, 7.9 Hz, 1H), 7.35 (dd, J=4.6, 8.0 Hz, 1H), 7.29 (s, 1H), 7.16 (d, J=0.7 Hz, 2H), 6.22 (s, 1H), 4.27 (d, J=12.1 Hz, 1H), 4.02 (br d, J=10.4 Hz, 1H), 3.9-3.8 (m, 1H), 3.77 (s, 3H), 3.72 (t, J=4.7 Hz, 4H), 3.67 (dd, J=1.7, 12.2 Hz, 1H), 3.5-3.4 (m, 1H), 3.26 (ddd, J=2.8, 6.7, 9.9 Hz, 1H), 2.89 (t, J=11.1 Hz, 1H), 2.8-2.6 (m, 2H), 2.50 (br s, 4H), 2.3-2.2 (m, 1H), 1.97 (br d, J=11.5 Hz, 4H), 1.6-1.6 (m, 4H), 1.25 (d, J=6.5 Hz, 3H).

### Example 28A and Example 28B

### 4-[(4R,10bS)-4-methyl-8-(endo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one and 4-[(4R,10bS)-4-methyl-8-(exo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one

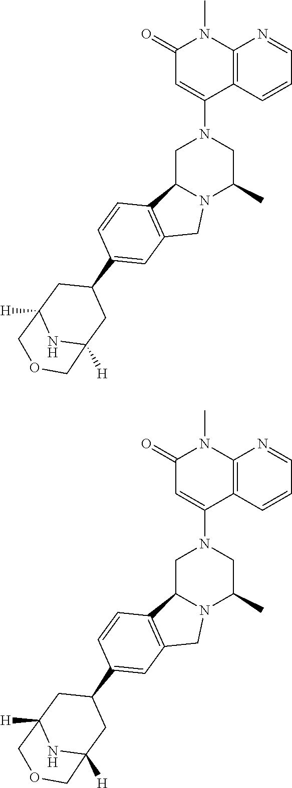 [SMILES: C[C@@H]1CN(c2cc(=O)n(C)c3ncccc23)C[C@@H]2c3ccc([C@H]4C[C@H]5COC[C@@H](C4)N5)cc3CN12 ; C[C@@H]1CN(c2cc(=O)n(C)c3ncccc23)C[C@@H]2c3ccc([C@@H]4C[C@H]5COC[C@@H](C4)N5)cc3CN12]

The titled compound was synthesized according to the following scheme:

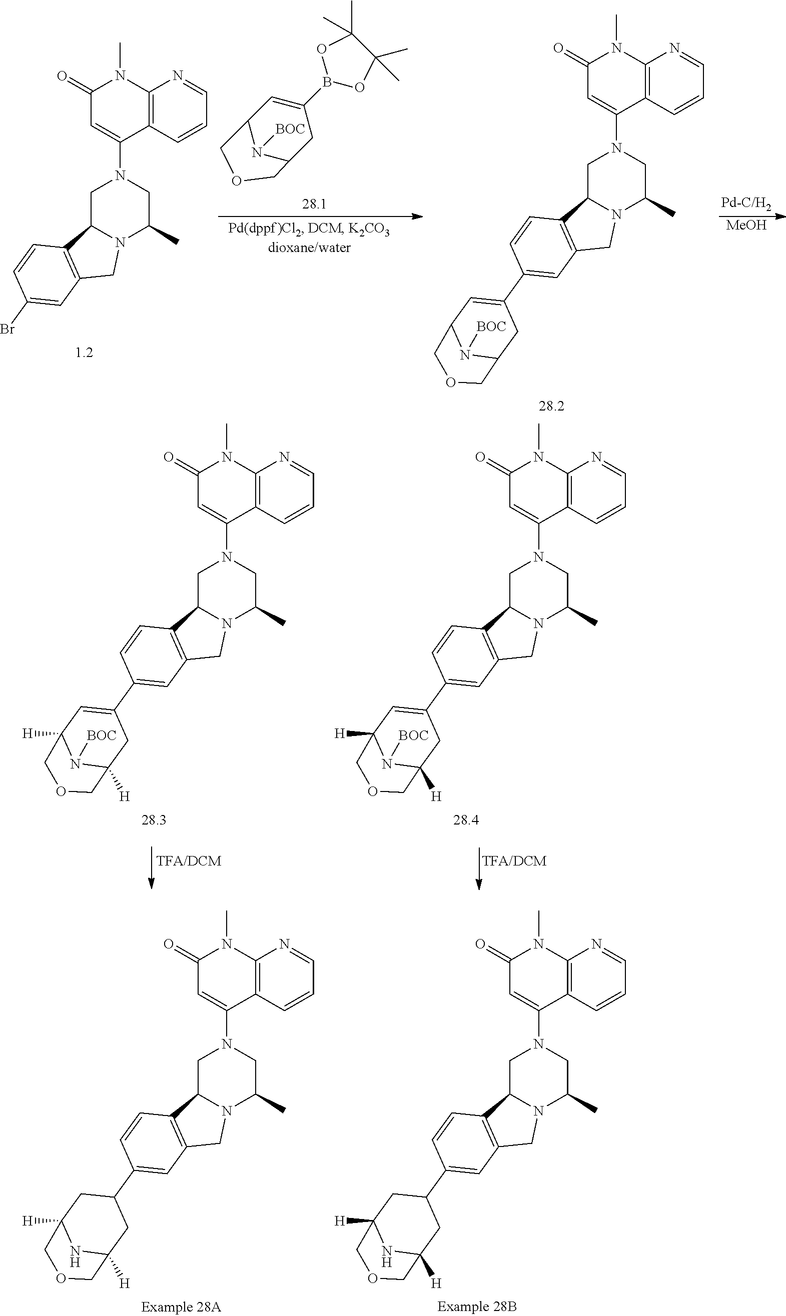

### Step 1: Preparation of tert-butyl 7-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]non-6-ene-9-carboxylate (compound 28.2)

To a solution of 4-[(4R,10bS)-8-bromo-4-methyl-3,4,6,10b-tetrahydro-1H-pyrazino[2,1-a]isoindol-2-yl]-1-methyl-1,8-naphthyridin-2-one (compound 1.2, 350 mg, 823 μmol) in dioxane (18 mL) and water (2 mL) was added tert-butyl 7-(4,4,5,5-tetramethyl-1,3,2-dioxaborolan-2-yl)-3-oxa-9-azabicyclo[3.3.1]non-6-ene-9-carboxylate (CAS: 1313034-29-5, PharmaBlock, Catalog: PB08083, 347 mg, 987 μmol), K₂CO₃ (227 mg, 1.65 mmol) and PdCl₂(dppf). DCM adduct (60.2 mg, 82.3 μmol). The resultant mixture was heated at 100° C. for 20 hrs. After being cooled to room temperature, diluted with water (30 mL) and extracted with DCM (60 mL) twice. The combined organic layer was washed with brine, dried over Na₂SO₄, filtered and concentrated in vacuo. The residue was purified by flash chromatography (silica gel, 40 g, 0% to 100% EtOAc in DCM) to afford compound 28.2 (310 mg, 75.6% yield). MS: calc'd 570 [(M+H)⁺], measured 570 [(M+H)⁺].

### Step 2: Preparation of tert-butyl 7-[(4R,10bS)-4-methyl-2-(endo-1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]nonane-9-carboxylate (compound 28.3) and tert-butyl 7-[(4R,10bS)-4-methyl-2-(exo-1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]nonane-9-carboxylate (compound 28.4)

A mixture of tert-butyl 7-[(4R,10bS)-4-methyl-2-(1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]nonane-9-carboxylate (compound 28.2, 310 mg, 544 μmol) and Pd—C (30 mg) in MeOH (50 mL) was hydrogenated by a hydrogen balloon at room temperature for 2 hours. After the catalyst was filtered off, the filtrate was concentrated in vacuo, which was resolved by SFC to give two single isomers: compound 28.3 (32 mg, 10.3% yield) and compound 28.4 (116 mg, 37.4% yield) with 50% Ethanol (0.1% NH₃H₂O)/CO₂ on OX (5 μm, 250×20 mm I.D.) column. MS: calc'd 572 [(M+H)⁺], measured 572 [(M+H)⁺]. The stereochemistry of compound 28.3 and compound 28.4 were determined by NOSEY.

### Step 3: Preparation of 4-[(4R,10bS)-4-methyl-8-(endo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one (Example 28A)

To a solution of tert-butyl 7-[(4R,10bS)-4-methyl-2-(endo-1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]nonane-9-carboxylate (compound 28.3, 30 mg, 52.4 μmol) in DCM (10 mL) was added TFA (5 mL). The reaction mixture was stirred at room temperature for 2 hrs, then concentrated to afford a crude product, which was purified by pre-HPLC to afford Example 28A (19 mg, 76.9% yield). MS: calc'd 472 [(M+H)⁺], measured 472 [(M+H)⁺]. ¹H NMR (400 MHz, METHANOL-d₄) δ ppm 8.68 (dd, J=1.6, 4.6 Hz, 1H), 8.36 (dd, J=1.7, 7.9 Hz, 1H), 7.48 (s, 1H), 7.46-7.41 (m, 1H), 7.41-7.34 (m, 2H), 6.28 (s, 1H), 4.99 (dd, J=3.5, 10.9 Hz, 1H), 4.79 (d, J=13.3 Hz, 1H), 4.43 (d, J=13.4 Hz, 1H), 4.21-3.92 (m, 7H), 3.78 (s, 3H), 3.68-3.57 (m, 3H), 3.12-2.94 (m, 2H), 2.29 (br dd, J=1.9, 8.3 Hz, 4H), 1.47 (d, J=6.6 Hz, 3H).

### Step 4: Preparation of 4-[(4R,10bS)-4-methyl-8-(exo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one (Example 28B)

To a solution of tert-butyl 7-[(4R,10bS)-4-methyl-2-(exo-1-methyl-2-oxo-1,8-naphthyridin-4-yl)-3,4,6,10b-tetrahydro-1H-pyrazino[2,1-a]isoindol-8-yl]-3-oxa-9-azabicyclo[3.3.1]nonane-9-carboxylate (compound 28.4, 40 mg, 69.9 μmol) in DCM (10 mL) was added TFA (5 mL). The reaction mixture was stirred at room temperature for 2 hrs, then concentrated to afford a crude product, which was purified by pre-HPLC to afford Example 28B (26 mg, 78.6% yield). MS: calc'd 472 [(M+H)⁺], measured 472 [(M+H)⁺]. ¹H NMR (400 MHz, METHANOL-d₄) δ ppm 8.80-8.53 (m, 1H), 8.43-8.19 (m, 1H), 7.47 (s, 1H), 7.39-7.28 (m, 3H), 6.26 (s, 1H), 4.70-4.56 (m, 2H), 4.16 (d, J=13.1 Hz, 1H), 3.99-3.92 (m, 1H), 3.90-3.82 (m, 4H), 3.78-3.65 (m, 3H), 3.78 (s, 3H), 3.61-3.52 (m, 1H), 3.14-3.03 (m, 1H), 3.00-2.87 (m, 2H), 2.53-2.29 (m, 2H), 2.22-1.94 (m, 2H), 1.39 (d, J=6.6 Hz, 3H).

### Example 29

### 4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-ethyl-1,8-naphthyridin-2-one

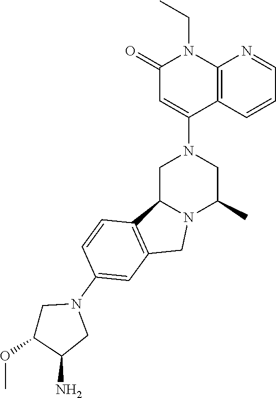 [SMILES: CCn1c(=O)cc(N2C[C@@H](C)N3Cc4cc(N5C[C@@H](N)[C@H](OC)C5)ccc4[C@H]3C2)c2cccnc21]

The titled compound was synthesized according to the following scheme:

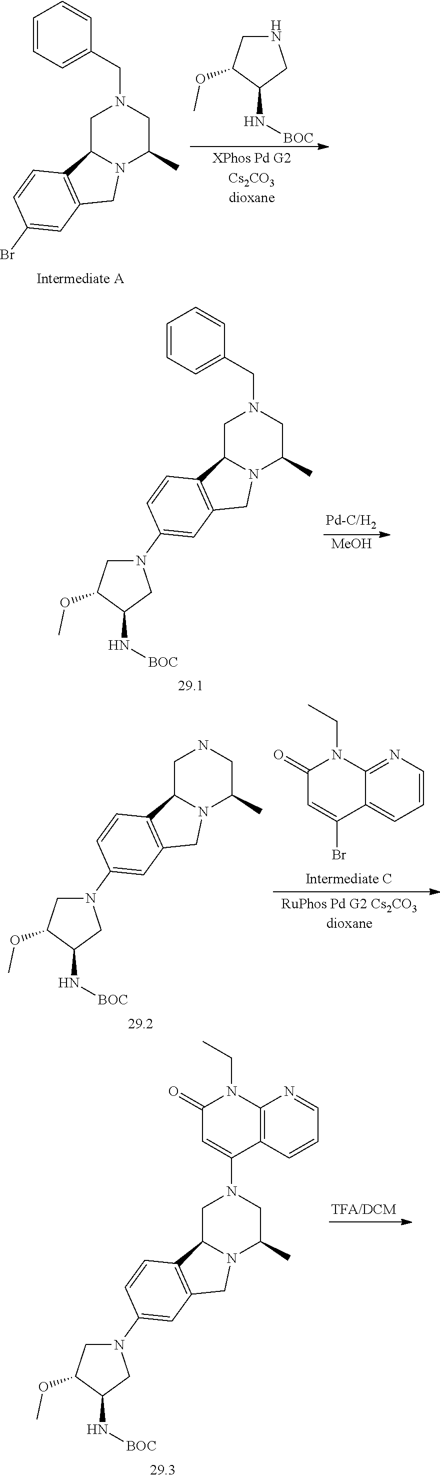

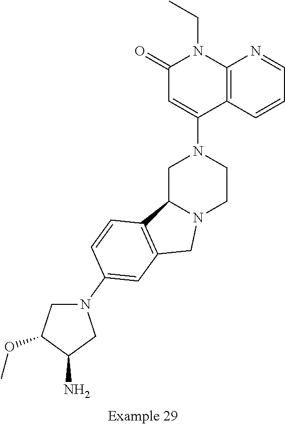 [SMILES: CCn1c(=O)cc(N2CCN3Cc4cc(N5C[C@@H](N)[C@H](OC)C5)ccc4[C@H]3C2)c2cccnc21]

### Step 1: Preparation of tert-butyl N-[(3R,4R)-1-[(4R,10bS)-2-benzyl-4-methyl-3,4,6,10b-tetrahydro-1H-pyrazino[2,1-a]isoindol-8-yl]-4-methoxy-pyrrolidin-3-yl]carbamate (compound 29.1)

To a solution of (4R,10bS)-2-benzyl-8-bromo-4-methyl-3,4,6,10b-tetrahydro-1H-pyrazino [2,1-a]isoindole (Intermediate A, 800 mg, 2.24 mmol) in dioxane (35 mL) was added tert-butyl ((3R,4R)-4-methoxypyrrolidin-3-yl)carbamate (508 mg, 2.35 mmol), Cs₂CO₃ (2.19 g, 6.72 mmol) and XPhos Pd G2 (176 mg, 224 μmol). The reaction mixture was stirred at 95° C. overnight. After being cooled to room temperature, the mixture was diluted with water (30 mL) and extracted with EA (30 mL) for three times. The combined organic layer was washed with brine, dried over Na₂SO₄, filtered and concentrated in vacuo. The residue was purified by flash chromatography (silica gel, 80 g, 50% to 100% EA in PE) to afford compound 29.1 (870 mg, 79% yield). MS: calc'd 493 [(M+H)⁺], measured 493 [(M+H)⁺].

### Step 2: Preparation of tert-butyl N-[(3R,4R)-1-[(4R,10bS)-4-methyl-1,2,3,4,6,10b-hexahydropyrazino[2,1-a]isoindol-8-yl]-4-methoxy-pyrrolidin-3-yl]carbamate (compound 29.2)

A mixture of tert-butyl N-[(3R,4R)-1-[(4R,10bS)-2-benzyl-4-methyl-3,4,6,10b-tetrahydro-1H-pyrazino[2,1-a]isoindol-8-yl]-4-methoxy-pyrrolidin-3-yl]carbamate (compound 29.1, 650 mg, 1.32 mmol) and Pd(OH)₂—C (100 mg) in MeOH (20 mL) was hydrogenated by a hydrogen balloon at room temperature for 2 hours. After the catalyst was filtered off, the filtrate was concentrated in vacuo to afford crude compound 29.2 (531 mg, 100% yield) which was used directly for the next step without further purification. MS: calc'd 403 [(M+H)⁺], measured 403 [(M+H)⁺].

### Step 3: Preparation of tert-butyl N-[(3R,4R)-1-[(4R,10bS)-2-(1-ethyl-2-oxo-1,8-naphthyridin-4-yl)-4-methyl-3,4,6,10b-tetrahydro-1H-pyrazino[2,1-a]isoindol-8-yl]-4-methoxy-pyrrolidin-3-yl]carbamate (compound 29.3)

To a solution of tert-butyl N-[(3R,4R)-1-[(4R,10bS)-4-methyl-1,2,3,4,6,10b-hexahydropyrazino[2,1-a]isoindol-8-yl]-4-methoxy-pyrrolidin-3-yl]carbamate (compound 29.2, 113 mg, 280 μmol) in dioxane (10 mL) was added 4-bromo-1-ethyl-1,8-naphthyridin-2-one (Intermediate C, 71 mg, 280 μmol), Cs₂CO₃ (274 mg, 840 μmol) and RuPhos Pd G2 (22 mg, 28 μmol). The reaction mixture was stirred at 95° C. overnight. After being cooled to room temperature, the mixture was diluted with water (30 mL) and extracted with EA (30 mL) for three times. The combined organic layer was washed with brine, dried over Na₂SO₄, filtered and concentrated in vacuo. The residue was purified by flash chromatography to afford compound 29.3 (30 mg, 18.6% yield). MS: calc'd 575 [(M+H)⁺], measured 575 [(M+H)⁺].

### Step 4: Preparation of 4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-ethyl-1,8-naphthyridin-2-one (Example 29)

To a solution of tert-butyl N-[(3R,4R)-1-[(4R,10bS)-2-(1-ethyl-2-oxo-1,8-naphthyridin-4-yl)-4-methyl-3,4,6,10b-tetrahydro-1H-pyrazino[2,1-a]isoindol-8-yl]-4-methoxy-pyrrolidin-3-yl]carbamate (compound 29.3, 30 mg, 52 μmol) in DCM (5 mL) was added 2,2,2-trifluoroacetic acid (2 mL). The reaction mixture was stirred at room temperature for 2 hrs, then concentrated to afford a crude product, which was purified by pre-HPLC to afford Example 29 (16 mg, 66.2% yield).). MS: calc'd 475 [(M+H)⁺], measured 475 [(M+H)⁺]. ¹H NMR (400 MHz, METHANOL-d₄) δ ppm 8.60 (dd, J=1.7, 4.6 Hz, 1H), 8.26 (dd, J=1.6, 8.1 Hz, 1H), 7.32-7.23 (m, 2H), 6.70 (s, 1H), 6.65-6.57 (m, 1H), 6.18 (s, 1H), 5.15-5.03 (m, 1H), 4.84-4.80 (m, 1H), 4.46 (q, J=7.0 Hz, 3H), 4.13 (br d, J=1.2 Hz, 1H), 4.06-3.99 (m, 1H), 3.86-3.81 (m, 1H), 3.77 (dd, J=5.9, 10.8 Hz, 1H), 3.60 (s, 2H), 3.37 (s, 4H), 3.25 (br d, J=3.3 Hz, 2H), 3.14-2.79 (m, 2H), 1.42 (d, J=6.6 Hz, 3H), 1.19 (t, J=7.0 Hz, 3H).

### Example 30

The following tests were carried out in order to determine the activity of the compounds of formula (I), (Ia) or (Ib) in HEK293-Blue-hTLR-7/8/9 cells assay.

HEK293-Blue-hTLR-7 Cells Assay:

A stable HEK293-Blue-hTLR-7 cell line was purchased from InvivoGen (Cat. #: hkb-htlr7, San Diego, Calif., USA). These cells were originally designed for studying the stimulation of human TLR7 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR7 cells with TLR7 ligands. Therefore the reporter expression was declined by TLR7 antagonist under the stimulation of a ligand, such as R848 (Resiquimod), for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR7 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620˜655 nm using a spectrophotometer. The signaling pathway that TLR7 activation leads to downstream NF-κB activation has been widely accepted, and therefore similar reporter assay was modified for evaluating TLR7 antagonist.

HEK293-Blue-hTLR-8 Cells Assay:

A stable HEK293-Blue-hTLR-8 cell line was purchased from InvivoGen (Cat. #: hkb-htlr8, San Diego, Calif., USA). These cells were originally designed for studying the stimulation of human TLR8 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR8 cells with TLR8 ligands. Therefore the reporter expression was declined by TLR8 antagonist under the stimulation of a ligand, such as R848, for incubation of 20 hrs. The cell culture supernatant SEAP reporter activity was determined using QUANTI-Blue™ kit (Cat. #: rep-qb1, Invivogen, San Diego, Ca, USA) at a wavelength of 640 nm, a detection medium that turns purple or blue in the presence of alkaline phosphatase.

HEK293-Blue-hTLR8 cells were incubated at a density of 250,000-450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 60 uM R848 in above DMEM, perform incubation under 37° C. in a CO₂ incubator for 20 hrs. Then 20 μL of the supernatant from each well was incubated with 180 μL Quanti-blue substrate solution at 37° C. for 2 hrs and the absorbance was read at 620-655 nm using a spectrophotometer. The signaling pathway that TLR8 activation leads to downstream NF-κB activation has been widely accepted, and therefore similar reporter assay was modified for evaluating TLR8 antagonist.

HEK293-Blue-hTLR-9 Cells Assay:

A stable HEK293-Blue-hTLR-9 cell line was purchased from InvivoGen (Cat. #: hkb-htlr9, San Diego, Calif., USA). These cells were originally designed for studying the stimulation of human TLR9 by monitoring the activation of NF-κB. A SEAP (secreted embryonic alkaline phosphatase) reporter gene was placed under the control of the IFN-β minimal promoter fused to five NF-κB and AP-1-binding sites. The SEAP was induced by activating NF-κB and AP-1 via stimulating HEK-Blue hTLR9 cells with TLR9 ligands. Therefore the reporter expression was declined by TLR9 antagonist under the stimulation of a ligand, such as ODN2006 (Cat. #: tlrl-2006-1, Invivogen, San Diego, Calif., USA), for incubation of 20 hrs. The cell culture supernatant SEAP reporter activity was determined using QUANTI-Blue™ kit (Cat. #: rep-qb1, Invivogen, San Diego, Calif., USA) at a wavelength of 640 nm, a detection medium that turns purple or blue in the presence of alkaline phosphatase.

HEK293-Blue-hTLR9 cells were incubated at a density of 250,000˜450,000 cells/mL in a volume of 170 μL in a 96-well plate in Dulbecco's Modified Eagle's medium (DMEM) containing 4.5 g/L glucose, 50 U/mL penicillin, 50 mg/mL streptomycin, 100 mg/mL Normocin, 2 mM L-glutamine, 10% (v/v) heat-inactivated fetal bovine serum with addition of 20 μL test compound in a serial dilution in the presence of final DMSO at 1% and 10 μL of 20 uM ODN2006 in above DMEM, perform incubation under 37° C. in a CO₂ incubator for 20 hrs. Then 20 μL of the supernatant from each well was incubated with 180 μL Quanti-blue substrate solution at 37° C. for 2 h and the absorbance was read at 620˜655 nm using a spectrophotometer. The signaling pathway that TLR9 activation leads to downstream NF-κB activation has been widely accepted, and therefore similar reporter assay was modified for evaluating TLR9 antagonist.

The compounds of formula (I) have TLR7 and/or TLR8 inhibitory activities (IC₅₀ value)<0.1 μM. Moreover, most compounds also have TLR9 inhibitory activity <0.4 μM. Activity data of the compounds of the present invention were shown in Table 1.

TABLE 1
The activity of the compounds of present invention
in HEK293-Blue-hTLR-7/8/9 cells assays

| Example | HEK/hTLR7 | HEK/hTLR8 | HEK/hTLR9 |
|---|---|---|---|
| No | IC₅₀ (nM) | IC₅₀ (nM) | IC₅₀ (nM) |
| 1 | 30.4 | 2.9 | 262 |
| 2 | 11.5 | 1.7 | 136 |
| 3 | 21.1 | 2.6 | 325 |
| 4 | 39.5 | 2.9 | 127 |
| 5 | 38.1 | 11.5 | 257 |
| 6 | 62 | 4.9 | 197 |
| 7 | 43.7 | 5.9 | 247 |
| 8 | 19.4 | 7.3 | 226 |
| 9 | 62.6 | 1.5 | 266 |
| 10 | 9.1 | 4 | 248 |
| 11 | 30.5 | 24.1 | 88 |
| 12 | 11.9 | 0.8 | 192 |
| 13 | 13 | 1.3 | 186 |
| 14 | 6.4 | 1 | 144 |
| 15 | 12 | 7.2 | 84 |
| 16 | 15.9 | 3.9 | 101 |
| 17 | 2.8 | <0.3 | 222 |
| 18 | 9.8 | 2.5 | 163 |
| 19 | 15.6 | 11.3 | 116 |
| 20 | 52.1 | 4.5 | 96 |
| 21 | 13.7 | 1.8 | 138 |
| 22 | 35.8 | 4.9 | 113 |
| 23 | 31 | 1 | 213 |
| 24 | 3.7 | 1.4 | 156 |
| 25 | 6.7 | 2.6 | 124 |
| 26 | 8.3 | 6.5 | 132 |
| 27A | 7 | 2 | 99 |
| 27B | 10 | 10 | 105 |
| 28A | 7.2 | 1.7 | 153 |
| 28B | 8.6 | 4.4 | 255 |
| 29 | 85.2 | 26.3 | 340 |

### Example 31

### Human Microsomal Stability Assay

Human liver microsomes (Cat. NO.: 452117, Corning, USA) were preincubated with test compound for 10 minutes at 37° C. in 100 mM potassium phosphate buffer, pH 7.4. The reactions were initiated by adding NADPH regenerating system. The final incubation mixtures contained 1 μM test compound, 0.5 mg/mL liver microsomal protein, 1 mM MgCl₂, 1 mM NADP, 1 unit/mL isocitric dehydrogenase and 6 mM isocitric acid in 100 mM potassium phosphate buffer, pH 7.4. After incubation times of 0, 3, 6, 9, 15 and 30 minutes at 37° C., 300 μL of cold ACN (including internal standard) was added to 100 μL incubation mixture to terminate the reaction. Following precipitation and centrifugation, 100 uL supernatant will be taken out and added 300 uL water.

The amount of compound remaining in the samples was determined by LC-MS/MS. Controls of no NADPH regenerating system at zero and 30 minutes were also prepared and analyzed. The results were categorized as: low (<7.0 mL/min/kg), medium (7.0-16.2 mL/min/kg) and high (16.2-23.2 mL/min/kg). Test results were summarized in Table 2.

TABLE 2
Human microsomal stability results

| Example | CL (h) |
|---|---|
| No | (mL/min/kg) |
| 2 | 9.5 |
| 4 | 6.3 |
| 5 | 7.8 |
| 7 | 9.4 |
| 9 | 8.8 |
| 11 | 6.9 |
| 12 | 6.2 |
| 13 | 7.9 |
| 15 | 8.0 |
| 16 | 8.5 |
| 18 | 6.2 |
| 19 | 6.2 |
| 20 | 6.2 |
| 21 | 6.4 |
| 23 | 6.2 |
| 24 | 6.2 |
| 25 | 6.2 |
| 26 | 9.8 |
| 27A | 7.3 |
| 28A | 9.2 |
| 29 | 6.4 |

### Example 32

hERG Channel Inhibition Assay

The hERG channel inhibition assay is a highly sensitive measurement that identifies compounds exhibiting hERG inhibition related to cardiotoxicity in vivo. The hERG K⁺ channels were cloned in humans and stably expressed in a CHO (Chinese hamster ovary) cell line. CHO_{hERG} cells were used for patch-clamp (voltage-clamp, whole-cell) experiments. Cells were stimulated by a voltage pattern to activate hERG channels and conduct I_{KhERG} Currents (rapid delayed outward rectifier potassium current of the hERG channel). After the cells were stabilized for a few minutes, the amplitude and kinetics of I_{KhERG} were recorded at a stimulation frequency of 0.1 Hz (6 bpm). Thereafter, the test compound was added to the preparation at increasing concentrations. For each concentration, an attempt was made to reach a steady-state effect, usually, this was achieved within 3-10 min at which time the next highest concentration was applied. The amplitude and kinetics of I_{KhERG} are recorded in each concentration of the drug which were compared to the control values (taken as 100%). (references: Redfern W S, Carlsson L, Davis A S, Lynch W G, MacKenzie I, Palethorpe S, Siegl P K, Strang I, Sullivan A T, Wallis R, Camm A J, Hammond T G. 2003; Relationships between preclinical cardiac electrophysiology, clinical QT interval prolongation and torsade de pointes for a broad range of drugs: evidence for a provisional safety margin in drug development. Cardiovasc. Res. 58:32-45, Sanguinetti M C, Tristani-Firouzi M. 2006; hERG potassium channels and cardiac arrhythmia. Nature 440:463-469, Webster R, Leishman D, Walker D. 2002; Towards a drug concentration effect relationship for QT prolongation and torsades de pointes. Curr. Opin. Drug Discov. Devel. 5:116-26). Results of hERG are given in Table 3.

TABLE 3
hERG results

| Example | hERG | hERG |
|---|---|---|
| No | IC₂₀ (μM) | IC₅₀ (μM) |
| 2 | >10 | >20 |
| 5 | >10 | >20 |
| 13 | 7.2 | >20 |
| 14 | >10 | >20 |
| 15 | >10 | >20 |
| 18 | >10 | >20 |
| 19 | >10 | >20 |
| 22 | >10 | >20 |
| 24 | >10 | >20 |

1. A compound of formula (I), 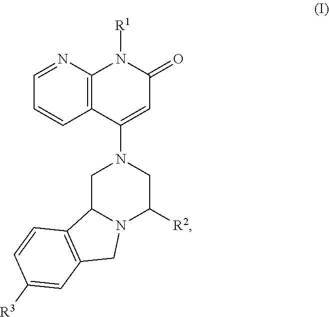
wherein
R¹ is C₁₋₆alkyl;
R² is C₁₋₆alkyl;
R³ is (C₁₋₆alkoxyC₁₋₆alkyl)piperazinyl; (C₁₋₆alkyl)₂aminoC₁₋₆alkoxy; 2,5-diazabicyclo[2.2.1]heptanyl; 3,4,4a,5,7,7a-hexahydro-2H-pyrrolo[3,4-b][1,4]oxazinyl; 3,8-diazabicyclo[3.2.1]octanyl; 3-oxa-9-azabicyclo[3.3.1]nonanyl; 5-oxa-2,8-diazaspiro[3.5]nonanyl; amino(C₁₋₆alkoxy)piperidinyl; amino(C₁₋₆alkoxy)pyrrolidinyl; amino(C₁₋₆alkyl)azetidinyl; amino(C₁₋₆alkyl)piperidinyl; amino(C₁₋₆alkyl)pyrrolidinyl; amino-1,4-oxazepanyl; aminohalopyrrolidinyl; aminopiperidinyl; C₁₋₆alkyl-2,6-diazaspiro[3.3]heptanyl; C₁₋₆alkylpiperazinyl; morpholinylC₃₋₇cycloalkyl; piperazinyl; piperidinyl or pyrrolidinyl;
or a pharmaceutically acceptable salt thereof.

2. A compound of formula (Ia), 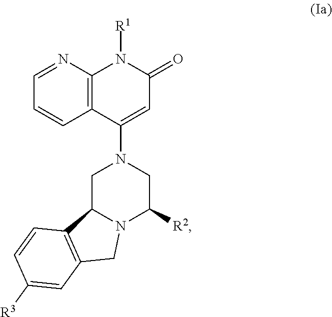
wherein
R¹ is C₁₋₆alkyl;
R² is C₁₋₆alkyl;
R³ is (C₁₋₆alkoxyC₁₋₆alkyl)piperazinyl; (C₁₋₆alkyl)₂aminoC₁₋₆alkoxy; 2,5-diazabicyclo[2.2.1]heptanyl; 3,4,4a,5,7,7a-hexahydro-2H-pyrrolo[3,4-b][1,4]oxazinyl; 3,8-diazabicyclo[3.2.1]octanyl; 3-oxa-9-azabicyclo[3.3.1]nonanyl; 5-oxa-2,8-diazaspiro[3.5]nonanyl; amino(C₁₋₆alkoxy)piperidinyl; amino(C₁₋₆alkoxy)pyrrolidinyl; amino(C₁₋₆alkyl)azetidinyl; amino(C₁₋₆alkyl)piperidinyl; amino(C₁₋₆alkyl)pyrrolidinyl; amino-1,4-oxazepanyl; aminohalopyrrolidinyl; aminopiperidinyl; C₁₋₆alkyl-2,6-diazaspiro[3.3]heptanyl; C₁₋₆alkylpiperazinyl; morpholinylC₃₋₇cycloalkyl; piperazinyl; piperidinyl or pyrrolidinyl;
or a pharmaceutically acceptable salt thereof.

3. A compound according to claim 1 or 2, wherein
R¹ is methyl or ethyl;
R² is methyl;
R³ is 2-(dimethylamino)ethoxy; 2-(methoxymethyl)piperazin-1-yl; 2,5-diazabicyclo[2.2.1]heptan-2-yl; 2-methylpiperazin-1-yl; 3,4,4a,5,7,7a-hexahydro-2H-pyrrolo[3,4-b][1,4]oxazin-6-yl; 3,8-diazabicyclo[3.2.1]octan-3-yl; 3-amino-1-piperidinyl; 3-amino-3-methyl-1-piperidinyl; 3-amino-3-methyl-azetidin-1-yl; 3-amino-3-methyl-pyrrolidin-1-yl; 3-amino-4-fluoro-pyrrolidin-1-yl; 3-amino-4-methoxy-1-piperidinyl; 3-amino-4-methoxy-pyrrolidin-1-yl; 3-methylpiperazin-1-yl; 3-oxa-9-azabicyclo[3.3.1]nonan-7-yl; 3-piperidinyl; 4-amino-3-methoxy-1-piperidinyl; 4-amino-4-methyl-1-piperidinyl; 4-morpholinocyclohexyl; 4-piperidinyl; 5-oxa-2,8-diazaspiro[3.5]nonan-2-yl; 5-oxa-2,8-diazaspiro[3.5]nonan-8-yl; 6-amino-1,4-oxazepan-4-yl; 6-methyl-2,6-diazaspiro[3.3]heptan-2-yl; piperazin-1-yl or pyrrolidin-3-yl;
or a pharmaceutically acceptable salt thereof.

4. A compound according to claim 1 or 2, wherein R³ is amino(C₁₋₆alkoxy)pyrrolidinyl; amino(C₁₋₆alkyl)piperidinyl; amino-1,4-oxazepanyl; aminopiperidinyl; C₁₋₆alkyl-2,6-diazaspiro[3.3]heptanyl; C₁₋₆alkylpiperazinyl; C₁₋₆alkylpiperazinyl; morpholinylC₃₋₇cycloalkyl; piperazinyl; piperidinyl or 3-oxa-9-azabicyclo[3.3.1]nonanyl.

5. A compound according to claim 4, wherein R³ is 3-amino-1-piperidinyl; 3-amino-3-methyl-1-piperidinyl; 3-amino-4-methoxy-pyrrolidin-1-yl; 3-methylpiperazin-1-yl; 4-amino-4-methyl-1-piperidinyl; 4-morpholinocyclohexyl; 4-piperidinyl; 6-amino-1,4-oxazepan-4-yl; 6-methyl-2,6-diazaspiro[3.3]heptan-2-yl; piperazin-1-yl or 3-oxa-9-azabicyclo[3.3.1]nonan-7-yl.

6. A compound according to claim 1 or 2, wherein
R¹ is C₁₋₆alkyl;
R² is C₁₋₆alkyl;
R³ is amino(C₁₋₆alkoxy)pyrrolidinyl; amino(C₁₋₆alkyl)piperidinyl; amino-1,4-oxazepanyl;
aminopiperidinyl; C₁₋₆alkyl-2,6-diazaspiro[3.3]heptanyl; C₁₋₆alkylpiperazinyl; C₁₋₆alkylpiperazinyl; morpholinylC₃₋₇cycloalkyl; piperazinyl; piperidinyl or 3-oxa-9-azabicyclo[3.3.1]nonanyl;
or a pharmaceutically acceptable salt thereof.

7. A compound according to claim 6, wherein
R¹ is methyl;
R² is methyl;
R³ is 3-amino-1-piperidinyl; 3-amino-3-methyl-1-piperidinyl 3-amino-4-methoxy-pyrrolidin-1-yl; 3-methylpiperazin-1-yl; 4-amino-4-methyl-1-piperidinyl; 4-morpholinocyclohexyl; 4-piperidinyl; 6-amino-1,4-oxazepan-4-yl; 6-methyl-2,6-diazaspiro[3.3]heptan-2-yl; piperazin-1-yl or 3-oxa-9-azabicyclo[3.3.1]nonan-7-yl;
or a pharmaceutically acceptable salt thereof.

8. A compound selected from:
4-[(4R,10bS)-8-(3-amino-3-methyl-azetidin-1-yl)-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(6-methyl-2,6-diazaspiro[3.3]heptan-2-yl)-3,4,6,10b-tetrahydro-1H-pyrazino[2,1-a]isoindol-2-yl]-1-methyl-1,8-naphthyridin-2-one;
4-[(4R,10bS)-4-methyl-8-(5-oxa-2,8-diazaspiro[3.5]nonan-2-yl)-3,4,6,10b-tetrahydro-1H-pyrazino[2,1-a]isoindol-2-yl]-1-methyl-1,8-naphthyridin-2-one;
4-[(4R,10bS)-8-[(3R)-3-amino-3-methyl-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(4aR,7aR)-3,4,4a,5,7,7a-hexahydro-2H-pyrrolo[3,4-b][1,4]oxazin-6-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R,4S)-3-amino-4-fluoro-pyrrolidin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3S,4S)-4-amino-3-methoxy-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3S,4S)-3-amino-4-methoxy-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(2S)-2-(methoxymethyl)piperazin-1-yl]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(5-oxa-2,8-diazaspiro[3.5]nonan-8-yl)-3,4,6,10b-tetrahydro-1H-pyrazino[2,1-a]isoindol-2-yl]-1-methyl-1,8-naphthyridin-2-one;
4-[(4R,10bS)-4-methyl-8-piperazin-1-yl-3,4,6,10b-tetrahydro-1H-pyrazino[2,1-a]isoindol-2-yl]-1-methyl-1,8-naphthyridin-2-one;
4-[(4R,10bS)-4-methyl-8-[(3S)-3-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-4-methyl-8-[(3R)-3-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-8-[(3S)-3-amino-3-methyl-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-8-[(3R)-3-amino-1-piperidyl]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-[(2R)-2-methylpiperazin-1-yl]-3,4,6,10b-tetrahydro-1H-pyrazino[2,1-a]isoindol-2-yl]-1-methyl-1,8-naphthyridin-2-one;
4-[(4R,10bS)-8-(4-amino-4-methyl-1-piperidyl)-4-methyl-3,4,6,10b-tetrahydro-1H-pyrazino[2,1-a]isoindol-2-yl]-1-methyl-1,8-naphthyridin-2-one;
4-[(4R,10bS)-8-[(1S,4S)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[(1R,4R)-2,5-diazabicyclo[2.2.1]heptan-2-yl]-4-methyl-3,4,6,10b-tetrahydro-1H-pyrazino[2,1-a]isoindol-2-yl]-1-methyl-1,8-naphthyridin-2-one;
4-[(4R,10bS)-8-(3,8-diazabicyclo[3.2.1]octan-3-yl)-4-methyl-3,4,6,10b-tetrahydro-1H-pyrazino[2,1-a]isoindol-2-yl]-1-methyl-1,8-naphthyridin-2-one;
4-[(4R,10bS)-8-[(6R)-6-amino-1,4-oxazepan-4-yl]-4-methyl-3,4,6,10b-tetrahydro-1H-pyrazino[2,1-a]isoindol-2-yl]-1-methyl-1,8-naphthyridin-2-one;
4-[(4R,10bS)-8-[2-(dimethylamino)ethoxy]-4-methyl-3,4,6,10b-tetrahydro-1H-pyrazino[2,1-a]isoindol-2-yl]-1-methyl-1,8-naphthyridin-2-one;
4-[(4R,10bS)-4-methyl-8-(4-piperidyl)-3,4,6,10b-tetrahydro-1H-pyrazino[2,1-a]isoindol-2-yl]-1-methyl-1,8-naphthyridin-2-one;
4-[(4R,10bS)-4-methyl-8-(3-piperidyl)-3,4,6,10b-tetrahydro-1H-pyrazino[2,1-a]isoindol-2-yl]-1-methyl-1,8-naphthyridin-2-one;
4-[(4R,10bS)-4-methyl-8-pyrrolidin-3-yl-3,4,6,10b-tetrahydro-1H-pyrazino[2,1-a]isoindol-2-yl]-1-methyl-1,8-naphthyridin-2-one;
4-[(4R,10bS)-4-methyl-8-(trans-4-morpholinocyclohexyl)-3,4,6,10b-tetrahydro-1H-pyrazino[2,1-a]isoindol-2-yl]-1-methyl-1,8-naphthyridin-2-one;
4-[(4R,10bS)-4-methyl-8-(cis-4-morpholinocyclohexyl)-3,4,6,10b-tetrahydro-1H-pyrazino[2,1-a]isoindol-2-yl]-1-methyl-1,8-naphthyridin-2-one;
4-[(4R,10bS)-4-methyl-8-(endo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one;
4-[(4R,10bS)-4-methyl-8-(exo-3-oxa-9-azabicyclo[3.3.1]nonan-7-yl)-3,4,6,10b-tetrahydro-1H-pyrazino[2,1-a]isoindol-2-yl]-1-methyl-1,8-naphthyridin-2-one; and
4-[(4R,10bS)-8-[(3R,4R)-3-amino-4-methoxy-pyrrolidin-1-yl]-4-methyl-3,4,6,10b-tetrahydro-1H-pyrazino[2,1-a]isoindol-2-yl]-1-ethyl-1,8-naphthyridin-2-one;
or a pharmaceutically acceptable salt, enantiomer or diastereomer thereof.

9. A process for the preparation of a compound according to any one of claims 1 to 8 comprising any of the following steps:
c) the substitution reaction or Buchwald-Hartwig amination of compound of formula (XII), 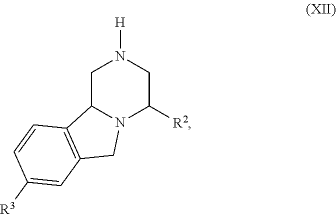
and compound of formula (XIII), (XIII); 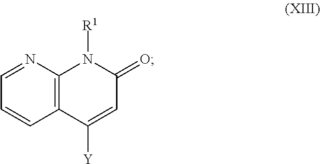
d) the Buchwald-Hartwig amination reaction of compound of formula (XV), 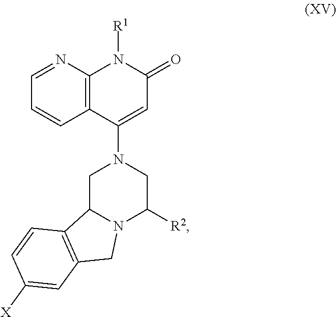
and amine HR³; or Suzuki coupling reaction between compound of formula (IX) and R³-boronic acid or R³-boronic ester;
wherein X is halogen; R¹ to R³ are defined as in any one of claims 1 to 7.

10. A compound or pharmaceutically acceptable salt, enantiomer or diastereomer according to any one of claims 1 to 8 for use as therapeutically active substance.

11. A pharmaceutical composition comprising a compound in accordance with any one of claims 1 to 8 and a therapeutically inert carrier.

12. The use of a compound according to any one of claims 1 to 8 for the treatment or prophylaxis of systemic lupus erythematosus or lupus nephritis.

13. The use of a compound according to any one of claims 1 to 8 for the preparation of a medicament for the treatment or prophylaxis of systemic lupus erythematosus or lupus nephritis.

14. The use of a compound according to any one of claims 1 to 8 as the TLR7 or TLR8 or TLR9 antagonist.

15. The use of a compound according to any one of claims 1 to 8 as the TLR7 and TLR8 and TLR9 antagonist.

16. The use of a compound according to any one of claims 1 to 8 for the preparation of a medicament for TLR7 and TLR8 and TLR9 antagonist.

17. A compound or pharmaceutically acceptable salt, enantiomer or diastereomer according to any one of claims 1 to 8 for the treatment or prophylaxis of systemic lupus erythematosus or lupus nephritis.

18. A compound or pharmaceutically acceptable salt, enantiomer or diastereomer according to any one of claims 1 to 8 when manufactured according to a process of claim 9.

19. A method for the treatment or prophylaxis of systemic lupus erythematosus or lupus nephritis, which method comprises administering a therapeutically effective amount of a compound as defined in any one of claims 1 to 8.
